(19) United States
(12) Patent Application Publication
(10) Pub. No.: US 2002/0165380 A1
(43) Pub. Date: Nov. 7, 2002

(54) DNA encoding a mammalian receptor (fb41a) and uses thereof

(76) Inventor: Jonathan A. Bard, Doylestown, PA (US)

Correspondence Address: John P. White, Cooper & Dunham LLP, 1185 Avenue of the Americas, New York, NY, 10036, US

(21) Appl. No.: 09/925,922

(22) Filed: Aug. 9, 2001

Related U.S. Application Data
(63) Continuation-in-part of application No. 09/210,279, filed on Dec. 10, 1998, now abandoned.

(51) Int. Cl. C07K 14/705; C07H 21/04; C12P 21/02; C12N 5/06

(52) U.S. Cl. 536/23.5; 530/350; 435/325; 435/69.1; 435/320.1

(57) ABSTRACT

This invention provides an isolated nucleic acid encoding a mammalian fb41a receptor, a purified mammalian fb41a receptor, vectors comprising isolated nucleic acid encoding a mammalian fb41a receptor, cells comprising such vectors, antibodies directed to a mammalian fb41a receptor, nucleic acid probes useful for detecting nucleic acid encoding a mammalian fb41a receptor, antisense oligonucleotides complementary to unique sequences of nucleic acid encoding a mammalian fb41a receptor, transgenic, nonhuman animals which express DNA encoding a normal or mutant mammalian fb41a receptor, methods of isolating a mammalian fb41a receptor, methods of treating an abnormality that is linked to the activity of the mammalian fb41a receptor, as well as methods of determining binding of compounds to mammalian fb41a receptors.

### BACKGROUND OF THE INVENTION

Throughout this application, various publications are referenced in parentheses by author and year. Full citations for these references may be found at the end of the specification immediately preceding the claims. The disclosure of these publications in their entireties are hereby incorporated by reference into this application to describe more fully the art to which this invention pertains.

Neuroregulators comprise a diverse group of natural products that subserve or modulate communication in the nervous system. They include, but are not limited to, neuropeptides, amino acids, biogenic amines, lipids and lipid metabolites, and other metabolic byproducts. Many of these neuroregulator substances interact with specific cell surface receptors which transduce signals from the outside to the inside of the cell. G-protein. coupled receptors (GPCRs) represent a major class of cell surface receptors with which many neurotransmitters interact to mediate their effects. GPCRs are predicted to have seven membrane-spanning domains and are coupled to their effectors via G-proteins linking receptor activation with intracellular biochemical sequelae such as stimulation of adenylyl cyclase. While the structural motifs that characterize a GPCR can be recognized in the predicted amino acid sequence of a novel receptor, the endogenous ligand that activates the GPCR cannot necessarily be predicted from its primary structure. Thus, a novel receptor sequence may be designated as an “orphan” GPCR when its function as a G-protein coupled receptor can be accurately predicted but its endogenous activating ligand cannot.

The fb41a receptor is such an orphan GPCR. Isolated from genomic DNA by reduced stringency homology cloning using probes designed from the seven transmembrane regions of the human Y4 receptor, fb41a encodes a novel GPCR of unknown function. Its closest relatives are other GPCRs, but none exhibits greater than 27% amino acid identity with fb41a. This level of identity is typically too low to be predictive of with respect to shared activating ligands. , However, the endogenous ligand for fb41a is likely to be a neurotransmitter since the fb41a receptor is present in several regions of the human brain. Using a homology screening approach to clone novel receptor genes, we describe here the isolation and characterization of a novel receptor clone which we have designated the fb41a receptor gene. The receptor encoded by the fb41a receptor gene will enable us to discover the endogenous activating ligand and confirm the function of a potentially important neuroregulator. It further enables us to examine the possibility of receptor diversity and the existence of multiple subtypes within this family of receptors. These could then serve as invaluable tools for drug design for pathophysiological conditions such as memory loss, depression, anxiety, epilepsy, pain, hypertension, locomotor problems, circadian rhythm disorders, eating/body weight disorders, sexual/reproductive disorders, nasal congestion, diarrhea, gastrointestinal and cardiovascular disorders.

### SUMMARY OF THE INVENTION

This invention provides an isolated nucleic acid encoding a mammalian fb41a receptor. In one embodiment, the mammalian fb41a receptor is a human fb41a receptor.

This invention provides a purified mammalian fb41a receptor protein.

This invention provides a vector comprising a nucleic acid encoding a mammalian, fb41a receptor. This invention also provides a vector comprising a nucleic acid encoding a human fb41a receptor. Such vector may be adapted for expression of the mammalian fb41a receptor in mammalian or non-mammalian cells.

This invention provides a plasmid designated FB41a (ATCC Accession No. 209449). This invention provides a cell comprising a vector which comprises a nucleic acid encoding a mammalian fb41a receptor. This invention also provides a membrane preparation isolated from such cells.

This invention provides a nucleic acid probe comprising at least 15 nucleotides, which probe specifically hybridizes with a nucleic acid encoding a mammalian fb41a receptor, wherein the probe has a unique sequence corresponding to a sequence present within one of the two strands of the nucleic acid encoding the mammalian fb41a receptor and are contained in plasmid FB41a (ATCC Accession No. 209449).

This invention further provides a nucleic acid probe comprising at least 15 nucleotides, which probe specifically hybridizes with a nucleic acid encoding a mammalian fb41a receptor, wherein the probe has a unique sequence corresponding to a sequence present within (a) the nucleic acid sequence shown in FIGS. 1A-1B(Seq. I.D. No. 1) or (b) the reverse complement thereto. This invention also provides a nucleic acid probe comprising a nucleic acid molecule of at least 15 nucleotides which is complementary to a unique fragment of the sequence of a nucleic acid molecule encoding a mammalian fb41a receptor.

This invention provides a nucleic acid probe comprising a nucleic acid molecule of at least 15 nucleotides which is complementary to the antisense sequence of a unique fragment of the sequence of a nucleic acid molecule encoding a mammalian fb41a receptor.

This invention provides an antisense oligonucleotide having a sequence capable of specifically hybridizing to the RNA of the mammalian fb41a receptor, so as to prevent translation of the RNA. This invention also provides an antisense oligonucleotide having a sequence capable of specifically hybridizing to the genomic DNA encoding a mammalian fb41a receptor.

This invention further provides an antibody capable of binding to a mammalian fb41a receptor. This invention also provides an agent capable of competitively inhibiting the binding of the antibody to a mammalian fb41a receptor.

This invention provides a pharmaceutical composition comprising (a) an amount of the oligonucleotide described above capable of passing through a cell membrane and effective to reduce expression of a mammalian fb41a receptor and (b) a pharmaceutically acceptable carrier capable of passing through the cell membrane.

This invention provides a transgenic, nonhuman mammal expressing DNA encoding a mammalian fb41a receptor. This invention also provides a transgenic, nonhuman mammal comprising a homologous recombination knockout of the native mammalian fb41a receptor. This invention further provides a transgenic, nonhuman mammal whose genome comprises antisense DNA complementary to the DNA encoding a mammalian fb41a receptor so placed within the genome as to be transcribed into antisense mRNA which is complementary to mRNA encoding the mammalian fb41a receptor and which hybridizes to mRNA encoding the mammalian fb41a receptor, thereby reducing its translation.

This invention provides a process for identifying a chemical compound which specifically binds to a mammalian fb41a receptor which comprises contacting cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor.

This invention provides a process for identifying a chemical compound which specifically binds to a mammalian fb41a receptor which comprises contacting a membrane fraction from a cell extract of cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor.

This invention provides a process involving competitive binding for identifying a chemical compound which specifically binds to a mammalian fb41a receptor which comprises separately contacting cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

This invention provide a process involving competitive binding for identifying a chemical compound which specifically binds to a mammalian fb41a receptor which comprises separately contacting a membrane fraction from a cell extract of cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

This invention provides a method of screening a plurality of chemical compounds not known to bind to a mammalian fb41a receptor to identify a compound which specifically binds to the mammalian fb41a receptor, which comprises (a) contacting cells transfected with and expressing DNA encoding the mammalian fb41a receptor with a compound known to bind specifically to the mammalian fb41a receptor; (b) contacting the preparation of step (a) with the plurality of compounds not known to bind specifically to the mammalian fb41a receptor, under conditions permitting binding of compounds known to bind the mammalian fb41a receptor; (c) determining whether the binding of the compound known to bind to the mammalian fb41a receptor is reduced in the presence of the compounds within the plurality of compounds, relative to the binding of the compound in the absence of the plurality of compounds; and if so (d) separately determining the binding to the mammalian fb41a receptor of compounds included in the plurality of compounds, so as to thereby identify the compound which specifically binds to the mammalian fb41a receptor.

This invention provides a method of screening a plurality of chemical compounds not known to bind to a mammalian fb41a receptor to identify a compound which specifically binds to the mammalian fb41a receptor, which comprises (a) preparing a cell extract from cells transfected with and expressing DNA encoding the mammalian fb41a receptor, isolating a membrane fraction from the cell extract, contacting the membrane fraction with a compound known to bind specifically to the mammalian fb41a receptor; (b) contacting the preparation of step (a) with the plurality of compounds not known to bind specifically to the mammalian fb41a receptor, under conditions permitting binding of compounds known to bind the mammalian fb41a receptor; (c) determining whether the binding of the compound known to bind to the mammalian fb41a receptor is reduced in the presence of the compounds within the plurality of compounds, relative to the binding of the compound in the absence of the plurality of compounds; and if so (d) separately determining the binding to the mammalian fb41a receptor of compounds included in the plurality of compounds, so as to thereby identify the compound which specifically binds to the mammalian fb41a receptor.

This invention provides a method of detecting expression of a mammalian fb41a receptor by detecting the presence of mRNA coding. for the mammalian fb41a receptor which comprises obtaining total mRNA from the cell and contacting the mRNA so obtained with a nucleic acid probe under hybridizing conditions, detecting the presence of mRNA hybridizing to the probe, and thereby detecting the expression of the mammalian fb41a receptor by the cell.

This invention provides a method of detecting the presence of a mammalian fb41a receptor on the surface of a cell which comprises contacting the cell with an antibody under conditions permitting binding of the antibody to the receptor, detecting the presence of the antibody bound to the cell, and thereby detecting the presence of the mammalian fb41a receptor on the surface of the cell.

This invention provides a method of determining the physiological effects of varying levels of activity of mammalian fb41a receptors which comprises producing a transgenic, nonhuman mammal whose levels of mammalian fb41a receptor activity are varied by use of an inducible promoter which regulates mammalian fb41a receptor expression.

This invention provides a method of determining the physiological effects of varying levels of activity of mammalian fb41a receptors which comprises producing a panel of transgenic, nonhuman mammals each expressing a different amount of mammalian fb41a receptor.

This invention provides a method for identifying an antagonist capable of alleviating an abnormality wherein the abnormality is alleviated by decreasing the activity of a mammalian fb41a receptor comprising administering a compound to the transgenic, nonhuman mammal and determining whether the compound alleviates the physical and behavioral abnormalities displayed by the transgenic, nonhuman mammal as a result of overactivity of a mammalian fb41a receptor, the alleviation of the abnormality identifying the compound as an antagonist. This invention also provides an antagonist identified by this method. This invention further provides a pharmaceutical composition comprising an antagonist identified by this method and a pharmaceutically acceptable carrier.

This invention provides a method of treating an abnormality in a subject wherein the abnormality is alleviated by decreasing the activity of a mammalian fb41a receptor which comprises administering to the subject an effective amount of this pharmaceutical composition, thereby treating the abnormality.

This invention provides a method for identifying an agonist capable of alleviating an abnormality in a subject wherein the abnormality is alleviated by increasing the activity of a mammalian fb41a receptor comprising administering a compound to a transgenic, nonhuman mammal, and determining whether the compound alleviates the physical and behavioral abnormalities displayed by the transgenic, nonhuman mammal, the alleviation of the abnormality identifying the compound as an agonist. This invention also provides an agonist identified by this method. This invention further provides a pharmaceutical composition comprising an agonist identified by this method and a pharmaceutically acceptable carrier. This invention provides a method of treating an abnormality in a subject wherein the abnormality is alleviated by increasing the activity of a mammalian fb41a receptor which comprises administering to the subject an effective amount of this pharmaceutical composition, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian fb41a receptor and labeled with a detectable marker; (e) detecting labeled bands which have hybridized to the DNA encoding a mammalian fb41a receptor labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

This invention provides a method of preparing a purified mammalian fb41a receptor which comprises: (a) inducing cells to express the mammalian fb41a receptor; (b) recovering the mammalian fb41a receptor from the induced cells; and (c) purifying the mammalian fb41a receptor so recovered.

This invention provides a method of preparing a purified mammalian fb41a receptor which comprises: (a)inserting nucleic acid encoding the mammalian fb41a receptor in a suitable vector; (b) introducing the resulting vector in a suitable host cell; (c) placing the resulting cell in suitable condition permitting the production of the isolated mammalian fb41a receptor; (d) recovering the mammalian fb41a receptor produced by the resulting cell; and (e) purifying the mammalian fb41a receptor so recovered.

This invention provides a process for determining whether a chemical compound is a mammalian fb41a receptor agonist which comprises contacting cells transfected with and expressing DNA encoding the mammalian fb41a receptor with the compound under conditions permitting the activation of the mammalian fb41a receptor, and detecting an increase in mammalian fb41a receptor activity, so as to thereby determine whether the compound is a mammalian fb41a receptor agonist. This invention also provides a pharmaceutical composition which comprises an amount of a mammalian fb41a receptor agonist determined by this process effective to increase activity of a mammalian fb41a receptor and a pharmaceutically acceptable carrier.

This invention provides a process for determining whether a chemical compound is a mammalian fb41a receptor antagonist which comprises contacting cells transfected with and expressing DNA encoding the mammalian fb41a receptor with the compound in the presence of a known mammalian fb41a receptor agonist, under conditions permitting the activation of the mammalian fb41a receptor, and detecting a decrease in mammalian fb41a receptor activity, so as to thereby determine whether the compound is a mammalian fb41a receptor antagonist. This invention also provides a pharmaceutical composition which comprises an amount of a mammalian fb41a receptor antagonist determined by this process effective to reduce activity of a mammalian fb41a receptor and a pharmaceutically acceptable carrier.

This invention provides a process for determining whether a chemical compound specifically binds to and activates a mammalian fb41a receptor, which comprises contacting cells producing a second messenger response and expressing on their cell surface the mammalian fb41a receptor, wherein such cells do not normally express the mammalian fb41a receptor, with the chemical compound under conditions suitable for activation of the mammalian fb41a receptor, and measuring the second messenger response in the presence and in the absence of the chemical compound, a change in the second messenger response in the presence of the chemical compound indicating that the compound activates the mammalian fb41a receptor. This invention also provides a compound determined by this process. This invention further provides a pharmaceutical composition which comprises an amount of the compound (a fb41a receptor agonist) determined by this process effective to increase activity of a mammalian fb41a receptor and a pharmaceutically acceptable carrier.

This invention provides a process for determining whether a chemical compound specifically binds to and inhibits activation of a mammalian fb41a receptor, which comprises separately contacting cells producing a second messenger response and expressing on their cell surface the mammalian fb41a receptor, wherein such cells do not normally express the mammalian fb41a receptor, with both the chemical compound and a second chemical compound known to activate the mammalian fb41a receptor, and with only the second chemical compound, under conditions suitable for activation of the mammalian fb41a receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian fb41a receptor. This invention also provides a compound determined by this process. This invention further provides a pharmaceutical composition which comprises an amount of the compound (a mammalian fb41a receptor antagonist) determined by this effective to reduce activity of a mammalian fb41a receptor and a pharmaceutically acceptable carrier.

This invention provides a method of screening a plurality of chemical compounds not known to activate a mammalian fb41a receptor to identify a compound which activates the mammalian fb41a receptor which comprises: (a) contacting cells transfected with and expressing the mammalian fb41a receptor with the plurality of compounds not known to activate the mammalian fb41a receptor, under conditions permitting activation of the mammalian fb41a receptor; (b) determining whether the activity of the mammalian fb41a receptor is increased in the presence of the compounds; and if so (c) separately determining whether the activation of the mammalian fb41a receptor is increased by each compound included in the plurality of compounds, so as to thereby identify the compound which activates the mammalian fb41a receptor. This invention also provides a compound identified by this method. This invention further provides a pharmaceutical composition which comprises an amount of the compound (a mammalian fb41a receptor agonist) identified by this method effective to increase activity of a mammalian fb41a receptor and a pharmaceutically acceptable carrier.

This invention provides a, method of screening a plurality of chemical compounds not known to inhibit the activation of a mammalian fb41a receptor to identify a compound which inhibits the activation of the mammalian fb41a receptor, which comprises: (a) contacting cells transfected with and expressing the mammalian fb41a receptor with the plurality of compounds in the presence of a known mammalian fb41a receptor agonist, under conditions permitting activation of the mammalian fb41a receptor; (b) determining whether the activation of the mammalian fb41a receptor is reduced in the presence of the plurality of compounds, relative to the activation of the mammalian fb41a receptor in the absence of the plurality of compounds; and if so (c) separately determining the inhibition of activation of the mammalian fb41a receptor for each compound included in the plurality of compounds, so as to thereby identify the compound which inhibits the activation of the mammalian fb41a receptor. This invention also provides a compound identified by this method. This invention further provides a pharmaceutical composition which comprises an amount of the compound (a mammalian fb41a receptor antagonist) identified by this process effective to decrease activity of a mammalian fb41a receptor and a pharmaceutically acceptable carrier.

This invention provides a method of treating an abnormality in a subject wherein the abnormality is alleviated by increasing the activity of a mammalian fb41a receptor which comprises administering to the subject an amount of a compound which is a mammalian fb41a receptor agonist effective to treat the abnormality.

This invention provides a method of treating an abnormality in a subject wherein the abnormality is alleviated by decreasing the activity of a mammalian fb41a receptor which comprises administering to the subject an amount of a compound which is a mammalian fb41a receptor antagonist effective to treat the abnormality.

### BRIEF DESCRIPTION OF THE FIGURES

FIGS. 1A-1B

Nucleotide sequence encoding a human receptor (fb41a) (Seq. I.D. No. 1). Two potential start (ATG) codons and the stop (TAA) codon are underlined.

FIGS. 2A-2C

Deduced amino acid sequence (Seq. I.D. No. 2) of the human receptor (fb41a) encoded by the nucleotide sequence shown FIGS. 1A-1B (Seq. I.D. No. 1). Seven solid lines designated I-VII.located above portions of the sequence indicate the seven putative transmembrane (TM) spanning regions.

FIG. 3

Partial coding sequence of rat receptor fb41a (SEQ. ID NO. 3).

FIG. 4

Partial amino acid sequence of the rat fb41a receptor (SEQ. ID NO. 4) encoded by the partial nucleotide sequence of FIG. 3.

FIG. 5

Comparison of rat and human nucleotide sequences for fb41a. Vertical lines indicate conserved residues.

FIG. 6

Autoradiograph demonstrating hybridization of radiolabeled rat fb41a probe to RNA extracted from rat tissue in a solution hybridization/nuclease protection assay using ³²P labeled riboprobe. 2 μg of mRNA was used in each assay. The single band (arrow) represents mRNA coding for the fb41a receptor extracted from the indicated tissue. Highest levels of mRNA coding for fb41a are found in: dorsal root ganglia, trigeminal ganglia, and neonatal brains. Integrity of RNA was assessed using hybridization to mRNA coding for GAPDH.

FIG. 7

Autoradiograph demonstrating hybridization of radiolabeled fb41a probe to RNA extracted from rat tissue in a solution hybridization/nucleiase protection assay. The bands (arrows) represent mRNA coding for the fb41a receptor extracted from the indicated tissue. Multiple bands representing fb41a mRNA are caused by splice variants of the mRNA or a hybridization artifact. Highest levels of mRNA coding for fb41a are found in fetal brain. Other areas expressing fb41a include: cerebellum, pituitary and substantia nigra. Integrity of RNA was assessed using hybridization to mRNA coding for GAPDH (not shown).

FIGS. 8A-8B

FIG. 8A: Hybridization of radiolabeled human fb41a riboprobe to a ZooBlot. Fb41a like gene sequences are present in several species including human, monkey, rat, dog, cow, rabbit, and yeast.

FIG. 8B: Hybridization of radiolabeled human fb41a riboprobe to a northern blot of fetal tissue. There is clear hybridization to mRNA extracted from fetal whole brain, with little or no specific hybridization in lung, liver or kidney.

### DETAILED DESCRIPTION OF THE INVENTION

Throughout this application, the following standard abbreviations are used to indicate specific nucleotide bases:

A=adenine

G=guanine

C=cytosine

T=thymine

U=uracil

M=adenine or cytosine

R=adenine or guanine

W=adenine, thymine, or uracil

S=cytosine or guanine

Y=cytosine, thymine, or uracil

K=guanine, thymine, or uracil

V=adenine, cytosine, or guanine (not thymine or uracil

H=adenine, cytosine, thymine, or uracil (not guanine)

D=adenine, guanine, thymine, or uracil (not cytosine)

B=cytosine, guanine, thymine, or uracil (not adenine)

N=adenine, cytosine, guanine, thymine, or uracil (or other modified base such as inosine)

I=inosine

Furthermore, the term agonist is used throughout this application to indicate any peptide or non-peptidyl compound which increases the activity of any of the polypeptides of the subject invention. The term antagonist is used throughout this application to indicate any peptide or non-peptidyl compound which decreases the activity of any of the polypeptides of the subject invention.

The activity of a G-protein coupled receptor such as the polypeptides disclosed herein may be measured using any of a variety of functional assays in which activation of the receptor in question results in an observable change in the level of some second messenger system, including, but not limited to, adenylate cyclase, calcium mobilization, arachidonic acid release, ion channel activity, inositol phospholipid hydrolysis or guanylyl cyclase. Heterologous expression systems utilizing appropriate host cells to express the nucleic acid of the subject invention are used to obtain the desired second messenger coupling. Receptor activity may also be assayed in an oocyte expression system.

It is possible that the mammalian fb41a receptor gene contains introns and furthermore, the possibility exists that additional introns could exist in coding or non-coding regions. In addition, spliced form(s) of mRNA may encode additional amino acids either upstream of the currently defined starting methionine or within the coding region. Further, the existence and use of alternative exons is possible, whereby the mRNA may encode different amino acids within the region comprising the exon. In addition, single amino acid substitutions may arise via the mechanism of RNA editing such that the amino acid sequence of the expressed protein is different than that encoded by the original gene. (Burns et al., 1996; Chu et al., 1996). Such variants may exhibit pharmacologic properties differing from the polypeptide encoded by the original gene.

This invention provides a splice variant of the mammalian fb41a receptor disclosed herein. This invention further provides for alternate translation initiation sites and alternately spliced or edited variants of nucleic acids encoding mammalian fb41a receptors of this invention.

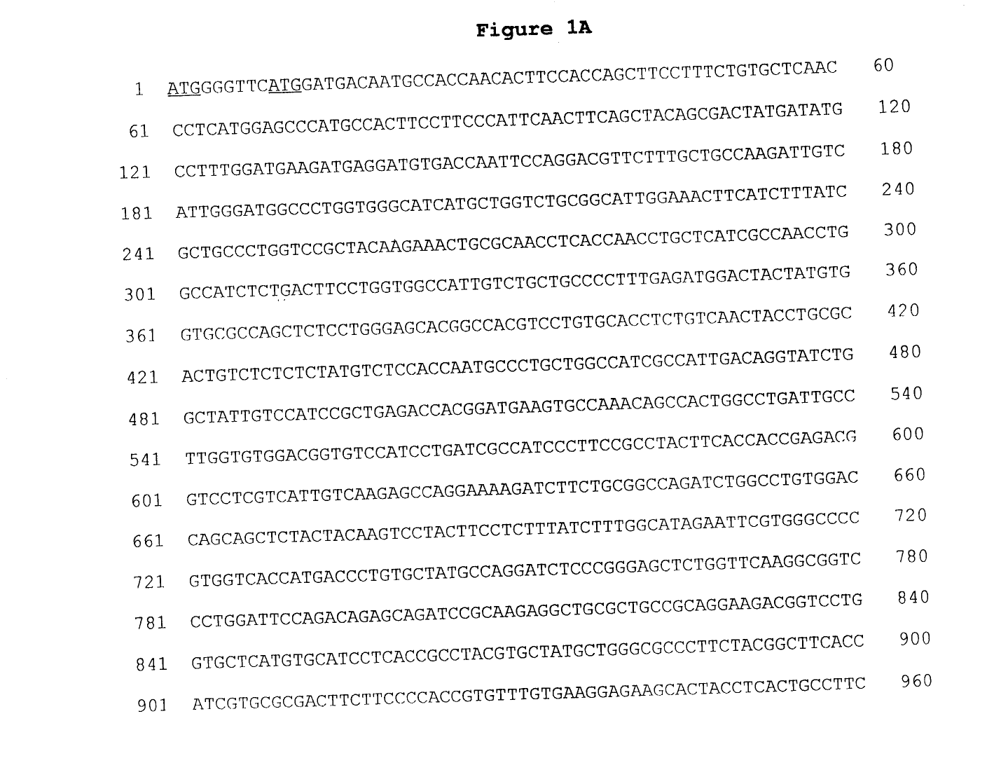

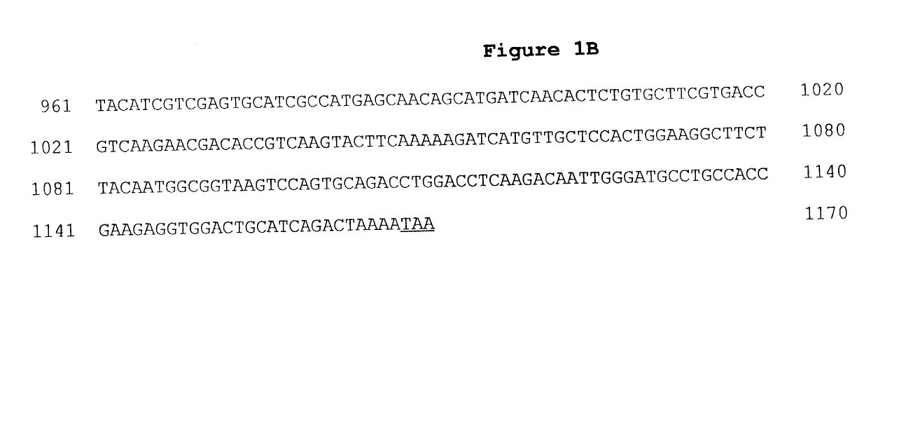

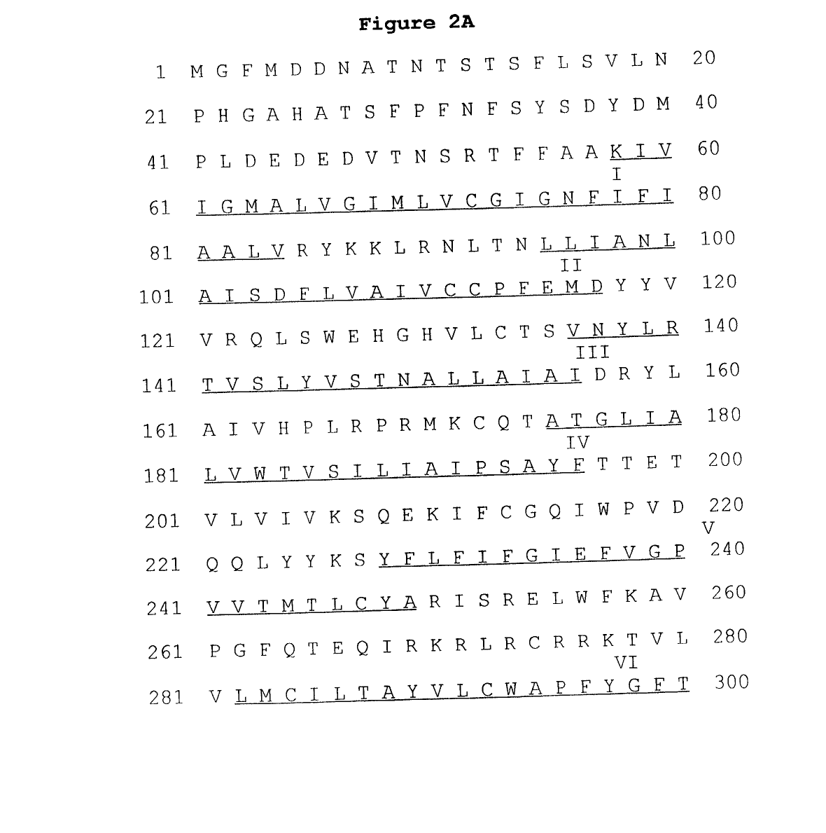

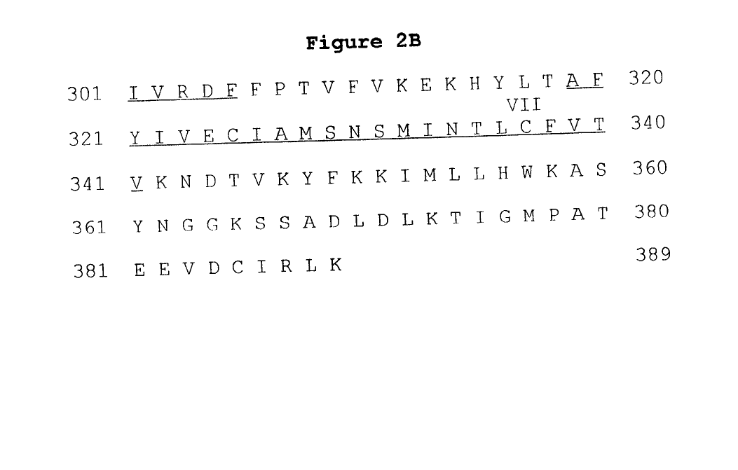

The nucleic acids of the subject invention also include nucleic acid analogs of the human fb41a receptor gene, wherein the human fb41a receptor gene comprises the nucleic acid sequence shown in FIG. 1A-lB or contained in plasmid FB41a (ATCC Accession No. 209449). A nucleic acid analog of the human fb41a receptor gene differs from the human fb41a receptor gene described herein in terms of the identity or location of one or more nucleic acid bases (deletion analogs containing less than all of the nucleic acid bases shown in FIGS. 1A-1B or contained in plasmid FB41a (ATCC Accession No. 209449), substitution analogs wherein one or more nucleic acid bases shown in FIG. 1A-1B or contained in plasmid FB41a (ATCC Accession No. 209449) are replaced by other nucleic acid bases, and addition analogs, wherein one or more nucleic acid bases are added to a terminal or medial portion of the nucleic acid sequence) and which encode proteins which share some or all of the properties of the protein encoded by the nucleic acid sequence shown in FIG. 1A-1B or contained in plasmid FB41a (ATCC Accession No. 209449). In one embodiment of the present invention, the nucleic acid analog encodes a protein which has an amino acid sequence identical to that shown in FIG. 2A-2C or encoded by the nucleic acid sequence contained in plasmid FB41a (ATCC Accession No. 209449). In another embodiment, the nucleic acid analog encodes a protein having an amino acid sequence which differs from the amino acid sequence shown in FIG. 2A-2C or encoded by the nucleic acid contained in plasmid FB41a (ATCC Accession No. 209449). In a further embodiment, the protein encoded by the nucleic acid analog has a function which is the same as the function of the receptor protein having the amino acid sequence shown in FIG. 2A-2C. In another embodiment, the function of the protein encoded by the nucleic acid analog differs from the function of the receptor protein having the amino acid sequence shown in FIG. 2A-2C. In separate embodiments, the variation in the nucleic acid sequence is less than 20 number of base pairs; preferably, less than 10 number of base pairs; more preferably, less than 5 number of base pairs. In another embodiment, the variation in the nucleic acid sequence occurs within the transmembrane (TM) region of the protein. In a further embodiment, the variation in the nucleic acid sequence occurs outside of the TM region.

This invention provides the above-described isolated nucleic acid, wherein the nucleic acid is DNA. In an embodiment, the DNA is CDNA. In another embodiment, the DNA is genomic DNA. In still another embodiment, the nucleic acid is RNA. Methods for production and manipulation of nucleic acid molecules are well known in the art.

This invention further provides nucleic acid which is degenerate with respect to the DNA encoding any of the polypeptides described herein. In an embodiment, the nucleic acid comprises a nucleotide sequence which is degenerate with respect to the nucleotide sequence shown in FIG. 1A-1B (SEQ ID NO. 2) or the nucleotide sequence contained in the plasmid FB41a (Accession No. 209449), that is, a nucleotide sequence which is translated into the same amino acid sequence.

This invention also encompasses DNAs and cDNAs which encode amino acid sequences which differ from those of the polypeptides of this invention, but which should not produce phenotypic changes. Alternately, this invention also encompasses DNAs, cDNAs, and RNAs which hybridize to the DNA, cDNA, and RNA of the subject invention. Hybridization methods are well known to those of skill in the art.

The nucleic acids of the subject invention also include nucleic acid molecules coding for polypeptide analogs, fragments or derivatives of antigenic polypeptides which differ from naturally-occurring forms in terms of the identity or location of one or more amino acid residues (deletion analogs containing less than all of the residues specified for the protein, substitution analogs wherein one or more residues specified are replaced by other residues and addition analogs wherein one or more amino acid residues is added to a terminal or medial portion of the polypeptides) and which share some or all properties of naturally-occurring forms. These molecules include: the incorporation of codons preferred for expression by selected non-mammalian hosts; the provision of sites for cleavage by restriction endonuclease enzymes; and the provision of additional initial, terminal or intermediate DNA sequences that facilitate construction of readily expressed vectors. The creation of polypeptide analogs is well known to those of skill in the art (R. F. Spurney et al. (1997); Fong, T. M. et al. (1995); Underwood, D. J. et al. (1994); Graziano, M. P. et al. (1996); Guam X. M. et al. (1995)).

The modified polypeptides of this invention may be transfected into cells either transiently or stably using methods well-known in the art, examples of which are disclosed herein. This invention also provides for binding assays using the modified polypeptides, in which the polypeptide is expressed either transiently or in stable cell lines. This invention further provides a compound identified using a modified polypeptide in a binding assay such as the binding assays described herein.

The nucleic acids described and claimed herein are useful for the information which they provide concerning the amino acid sequence of the polypeptide and as products for the large scale synthesis of the polypeptides by a variety of recombinant techniques. The nucleic acid molecule is useful for generating new cloning and expression vectors, transformed and transfected prokaryotic and eukaryotic host cells, and new and useful methods for cultured growth of such host cells capable of expression of the polypeptide and related products.

This invention provides an isolated nucleic acid encoding a mammalian fb41a receptor. In one embodiment, the nucleic acid is DNA. In another embodiment, the DNA is cDNA. In another embodiment, the DNA is genomic DNA. In another embodiment, the nucleic acid is RNA.

This invention further provides an isolated nucleic acid encoding a human fb41a receptor analog.

In one embodiment of the present invention, the mammalian fb41a receptor is a human fb41a receptor.

This invention also provides an isolated nucleic acid encoding a species homolog of the human fb41a receptor. In one embodiment, the nucleic acid encodes a mammalian fb41a receptor homolog which has substantially the same amino acid sequence as does the human fb41a receptor encoded by the plasmid FB41a (ATCC Accession No. 209449). In another embodiment, the nucleic acid encodes a mammalian FB41a receptor homolog which has about 65% amino acid identity to the human fb41a receptor encoded by the plasmid FB41a (ATCC Accession No. 209449). In a further embodiment, the nucleic acid encodes a mammalian fb41a receptor which has about 75% amino acid identity to the human fb41a receptor encoded by the plasmid FB41a (ATCC Accession No. 209449). In another embodiment, the nucleic acid encodes a mammalian fb41a receptor which has about 85% amino acid identity to the human fb41a receptor encoded by the plasmid FB41a (ATCC Accession No. 209449). In a further embodiment, the nucleic acid encodes a mammalian fb41a receptor which has about 95% amino acid identity to the human fb41a receptor encoded by the plasmid FB41a (ATCC Accession No. 209449). In a further embodiment, the nucleic acid encodes a mammalian fb41a receptor homolog which has an amino acid sequence identical to that of the human fb41a receptor encoded by the plasmid FB41a (ATCC Accession No. 209449). In another embodiment, the mammalian fb41a receptor homolog has about 70% nucleic acid identity to the human fb41a receptor gene contained in plasmid FB41a (ATCC Accession No. 209449). In a further embodiment, the mammalian fb41a receptor homolog has about 80% nucleic acid identity to the human fb41a receptor gene contained in the plasmid FB41a (ATCC Accession No. 209449). In another embodiment, the mammalian fb41a receptor homolog has about 90% nucleic acid identity to the human fb41a receptor gene contained in the plasmid FB41a (ATCC Accession No. 209449). In a further embodiment, the mammalian fb41a receptor homolog has about 100% nucleic acid identity to the human fb41a receptor gene contained in the plasmid FB41a (ATCC Accession No. 209449). Examples of methods for isolating and purifying species homologs are described elsewhere (U.S. Pat. No. 5,602,024) and below.

For example, once a human receptor gene has been cloned, oligonucleotide probes derived from the human gene sequence may be used to screen a genomic library in A dash II. The oligonucleotide is labeled with ³²P using polynucleotide kinase. Hybridization may be performed at medium stringency conditions: 45° C. in a solution containing 37.5%.formamide, 5× SSC (1× SSC in 0.5M NaCl, 0.015M sodium citrate), 1× Denhardt's solution (0.02% polyvinylpyrrolindone, 0.02% Ficoll, 0.02% BSA), and 200 μg/μl sonicated salmon sperm DNA. The filters are washed at 45° C. in 0.1× SSC containing 0.1% SDS and exposed at −70° C. to Kodak XAR film in the presence of an intensifying screen. Lambda phage clones hybridizing with the probe are plaque purified and DNA prepared for Southern blot analysis (Southern, 1975; Sambrook et al., 1989). A hybridizing fragment may be subcloned into a vector such as pUC18 (Pharmacia, Piscataway, N.J.). Nucleotide sequence analysis may be determined using standard procedures. The hybridizing fragment isolated above may be amplified using PCR with appropriate primers. The PCR primers are used to amplify single stranded cDNA prepared from brain as previously described. The amplified DNA is subcloned and sequenced.

A cDNA clone may also be isolated by screening pools of a cDNA library by PCR with appropriate primers. Positive pools identified may be analyzed further by sib selection to isolate a cDNA clone. DS-DNA may be sequenced as described above and nucleotide and peptide sequence analysis performed with GCG programs. For transient expression, COS-7 cells maybe transfected by the DEAE-Dextran method using 1 μg of DNA/10⁶ cells, as described elsewhere.

In one embodiment, the nucleic acid encodes a human fb41a receptor which has an amino acid sequence identical to that encoded by the plasmid FB41a (ATCC Accession No. 209449). In a further embodiment, the human fb41a receptor has a sequence substantially the same as the amino acid sequence shown in FIGS. 2A-2C (Seq. I.D. No. 2). In another embodiment, the human fb41a receptor has an amino acid sequence identical to the amino acid sequence shown in FIGS. 2A-2C (Seq. I.D. No. 2).

This invention provides an isolated nucleic acid encoding a modified mammalian fb41a receptor, which differs from a mammalian fb41a receptor by having an amino acid(s) deletion, replacement, or addition in the third intracellular domain.

This invention provides a purified mammalian fb41a receptor protein. In one embodiment, the purified mammalian fb41a receptor protein is a human fb41a receptor protein.

This invention provides a vector comprising the nucleic acid encoding a mammalian fb41a receptor. In another embodiment, the mammalian fb41a receptor is a human fb41a receptor.

In an embodiment, the vector is adapted for expression in a bacterial cell which comprises the regulatory elements necessary for expression of the nucleic acid in the bacterial cell operatively linked to the nucleic acid encoding the mammalian fb41a receptor as to permit expression thereof. In another embodiment, the vector is adapted for expression in an amphibian cell which comprises the regulatory elements necessary for expression of the nucleic acid in the amphibian cell operatively linked to the nucleic acid encoding the mammalian fb41a receptor as to permit expression thereof. In a further embodiment, the vector is adapted for expression in a yeast cell which comprises the regulatory elements necessary for expression of the nucleic acid in the yeast cell operatively linked to the nucleic acid encoding the mammalian fb41a receptor so as to permit expression thereof. In an embodiment, the vector is adapted for expression in an insect cell which comprises the regulatory elements necessary for expression of the nucleic acid in the insect cell operatively linked to the nucleic acid encoding the mammalian fb41a receptor so as to permit expression thereof. In another embodiment, the vector is a baculovirus. In a further embodiment, the vector is adapted for expression in a mammalian cell which comprises the regulatory elements necessary for expression of the nucleic acid in the mammalian cell operatively linked to the nucleic acid encoding the mammalian fb41a receptor so as to permit expression thereof. In one embodiment, the vector is a plasmid.

This invention provides a plasmid designated FB41a (ATCC Accession No. 209449). This plasmid comprises the regulatory elements necessary for expression of DNA in a mammalian cell operatively linked to DNA encoding the mammalian fb41a receptor so as to permit expression thereof.

This plasmid (FB41a) was deposited on Nov. 11, 1997, with the American Type Culture Collection (ATCC), 10801 University Blvd., Manassas, Virginia, U.S.A. under the provisions of the Budapest Treaty for the International Recognition of the Deposit of Microorganisms for the Purposes of Patent Procedure and was accorded ATCC Accession No. 209449.

This invention further provides for any vector or plasmid which comprises modified untranslated sequences, which are beneficial for expression in desired host cells or for use in binding or functional assays. For example, a vector or plasmid with untranslated sequences of varying lengths may express differing amounts of the polypeptide depending upon the host cell used. In an embodiment, the vector or plasmid comprises the coding sequence of the polypeptide and the regulatory elements necessary for expression in the host cell.

This invention provides a cell comprising a vector comprising a nucleic acid encoding the mammalian fb41a receptor. In an embodiment, the cell is a non-mammalian cell. In a further embodiment, the non-mammalian cell is a Xenopus oocyte cell or a Xenopus melanophore cell. In another embodiment, the cell is a mammalian cell. In a further embodiment, the mammalian cell is a COS-7 cell, a 293 human embryonic kidney cell, a NIH-3T3 cell, a LM(tk−) cell, a mouse Y1 cell, or a CHO cell.

This invention provides an insect cell comprising a vector adapted for expression in an insect cell which comprises a nucleic acid encoding a mammalian fb41a receptor. In another embodiment, the insect cell is an Sf9 cell, an Sf21 cell or a HighFive cell.

This invention provides a membrane preparation isolated from any one of the cells described above.

This invention provides a nucleic acid probe comprising at least 15 nucleotides, which probe specifically hybridizes with a nucleic acid encoding a mammalian fb41a receptor, wherein the probe has a unique sequence corresponding to a sequence present within one of the two strands of the nucleic acid encoding the mammalian fb41a receptor and are contained in plasmid fb41a (ATCC Accession No. 209449). This invention also provides a nucleic acid probe comprising at least 15 nucleotides, which probe specifically hybridizes with a nucleic acid encoding a mammalian fb41a receptor, wherein the probe has a unique sequence corresponding to a sequence present within (a) the nucleic acid sequence shown in FIG. 1 (Seq. I.D. No. 1) or (b) the reverse complement thereto. In one embodiment the nucleic acid is DNA. In another embodiment, the nucleic acid is RNA.

This invention provides a nucleic acid probe comprising a nucleic acid molecule of at least 15 nucleotides which is complementary to a unique fragment of the sequence of a nucleic acid molecule encoding a mammalian fb41a receptor. This invention also provides a nucleic acid probe comprising a nucleic acid molecule of at least 15 nucleotides which is complementary to the antisense sequence of a unique fragment of the sequence of a nucleic acid molecule encoding a mammalian fb41a receptor.

As used herein, the phrase specifically hybridizing means the ability of a nucleic acid molecule to recognize a nucleic acid sequence complementary to its own and to form double-helical segments through hydrogen bonding between complementary base pairs.

Nucleic acid probe technology is well known to those skilled in the art who will readily appreciate that such probes may vary greatly in length and may be labeled with a detectable label, such as a radioisotope or flourescent dye, to facilitate detection of the probe. DNA probe molecules may be produced by insertion of a DNA molecule which encodes the polypeptides of this invention into suitable vectors, such as plasmids or bacteriophages, followed by transforming into suitable bacterial host cells, replication in the transformed bacterial host cells and harvesting of the DNA probes, using methods well known in the art. Alternatively, probes may be generated chemically from DNA synthesizers.

RNA probes may be generated by inserting the DNA molecule which encodes the polypeptides of this invention downstream of a bacteriophage promoter such as T3, T7, or SP6. Large amounts of RNA probe may be produced by incubating the labeled nucleotides with the linearized fragment where it contains an upstream promoter in the presence of the appropriate RNA polymerase.

This invention provides an antisense oligonucleotide having a sequence capable of specifically hybridizing to RNA encoding a mammalian fb41a receptor, so as to prevent translation of the RNA. This invention also provides an antisense oligonucleotide having a sequence capable of specifically hybridizing to genomic DNA encoding a mammalian fb41a receptor. In one embodiment, the oligonucleotide comprises chemically modified nucleotides or nucleotide analogues.

This invention provides an antibody capable of binding to a mammalian fb41a receptor encoded by a nucleic acid encoding a mammalian fb41a receptor. In one embodiment, the mammalian fb41a receptor is a human fb41a receptor. This invention also provides an agent capable of competitively inhibiting the binding of the antibody to a mammalian fb41a receptor. In one embodiment, the antibody is a monoclonal antibody or antisera.

This invention provides a pharmaceutical composition comprising (a) an amount of the oligonucleotide capable of passing through a cell membrane and effective to reduce expression of a mammalian fb41a receptor and (b) a pharmaceutically acceptable carrier capable of passing through the cell membrane. In an embodiment, the oligonucleotide is coupled to a substance which inactivates mRNA. In a further embodiment, the substance which inactivates mRNA is a ribozyme. In another embodiment, the pharmaceutically acceptable carrier comprises a structure which binds to a mammalian fb41a receptor on a cell capable of being taken up by the cells after binding to the structure. In a further embodiment, the pharmaceutically acceptable carrier is capable of binding to a mammalian fb41a receptor which is specific for a selected cell type.

This invention provides a pharmaceutical composition which comprises an amount of an antibody effective to block binding of a ligand to a human fb41a receptor and a pharmaceutically acceptable carrier.

As used herein, the phrase pharmaceutically acceptable carrier means any of the standard pharmaceutically acceptable carriers. Examples include, but are not limited to, phosphate buffered saline, physiological saline, water, and emulsions, such as oil/water emulsions.

This invention provides a transgenic, nonhuman mammal expressing DNA encoding a mammalian fb41a receptor. This invention also provides a transgenic, nonhuman mammal comprising a homologous recombination knockout of the native mammalian fb41a receptor. This invention further provides a transgenic, nonhuman mammal whose genome comprises antisense DNA complementary to the DNA encoding a mammalian fb41a receptor so placed within the genome as to be transcribed into antisense mRNA which is complementary to mRNA encoding the mammalian fb41a receptor and which hybridizes to mRNA encoding the mammalian fb41a receptor, thereby reducing its translation. In an embodiment, the DNA encoding the mammalian fb41a receptor additionally comprises an inducible promoter. In another embodiment, the DNA encoding the mammalian fb41a receptor additionally comprises tissue specific regulatory elements. In a further embodiment, the transgenic, nonhuman mammal is a mouse.

Animal model systems which elucidate the physiological and behavioral roles of the polypeptides of this invention are produced by creating transgenic animals in which the activity of the polypeptide is either increased or decreased, or the amino acid sequence of the expressed polypeptide is altered, by a variety of techniques. Examples of these techniques include, but are not limited to: 1) Insertion of normal or mutant versions of DNA encoding the polypeptide, by microinjection, electroporation, retroviral transfection or other means well known to those in the art, into appropriate fertilized embryos in order to produce a transgenic animal or 2) Homologous recombination of mutant or normal, human or animal versions of these genes with the native gene locus in transgenic animals to alter the regulation of expression or the structure of these polypeptide sequences. The technique of homologous recombination is well known in the art. It replaces the native gene with the inserted gene and so is useful for producing an animal that cannot express native polypeptides but does express, for example, an inserted mutant polypeptide, which has replaced the native polypeptide in the animal's genome by recombination, resulting in underexpression of the transporter. Microinjection adds genes to the genome, but does not remove them, and so is useful for producing an animal which expresses its own and added polypeptides, resulting in overexpression of the polypeptides.

One means available for producing a transgenic animal, with a mouse as an example, is as follows: Female mice are mated, and the resulting fertilized eggs are dissected out of their oviducts. The eggs are stored in an appropriate medium such as M2 medium. DNA or cDNA encoding a polypeptide of this invention is purified from a vector by methods well known in the art. Inducible promoters may be fused with the coding region of the DNA to provide an experimental means to regulate expression of the trans-gene. Alternatively, or in addition, tissue specific regulatory elements may be fused with the coding region to permit tissue-specific expression of the trans-gene. The DNA, in an appropriately buffered solution, is put into a microinjection needle (which may be made from capillary tubing using a pipet puller) and the egg to be injected is put in a depression slide. The needle is inserted into the pronucleus of the egg, and the DNA solution is injected. The injected egg is then transferred into the oviduct of a pseudopregnant mouse (a mouse stimulated by the appropriate hormones to maintain pregnancy but which is not actually pregnant ), where it proceeds to the uterus, implants, and develops to term. As noted above, microinjection is not the only method for inserting DNA into the egg cell, and is used here only for exemplary purposes.

This invention provides a process for identifying a chemical compound which specifically binds to a mammalian fb41a receptor which comprises contacting cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor. This invention also provides a process for identifying a chemical compound which specifically binds to a mammalian fb41a receptor which comprises contacting a membrane fraction from a cell extract of cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor. In one embodiment, the mammalian fb41a receptor is a human fb41a receptor. In another embodiment, the mammalian fb41a receptor has substantially the same amino acid sequence as the mammalian fb41a receptor encoded by plasmid FB41a (ATCC Accession No. 209449). In a further embodiment, the mammalian fb41a receptor has substantially the same amino acid sequence as that shown in FIGS. 2A-2C (Seq. I.D. No. 2). In another embodiment, the mammalian fb41a receptor has the amino acid sequence shown in FIGS. 2A-2C (Seq. I.D. No. 2). In one embodiment, the compound is not previously known to bind to a mammalian fb41a receptor. This invention further provides a compound identified by the above-described process.

In one embodiment of the above-described processes, the cell is an insect cell. In another embodiment, the cell is a mammalian cell. In a further embodiment, the cell is nonneuronal in origin. In a further embodiment, the nonneuronal cell is a COS-7 cell, 293 human embryonic kidney cell, a CHO cell, a NIH-3T3 cell, a mouse Y1 cell, or a LM(tk−) cell. In an embodiment, the compound is a compound not previously known to bind to a mammalian fb41a receptor. This invention also provides a compound identified by the above-described process.

This invention provides a process involving competitive binding for identifying a chemical compound which specifically binds to a mammalian fb41a receptor which comprises separately contacting cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

This invention also provides a process involving competitive binding for identifying a chemical compound which specifically binds to a mammalian fb41a receptor which comprises separately contacting a membrane fraction from a cell extract of cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

In one embodiment, the mammalian fb41a receptor is a human fb41a receptor. In another embodiment, the human fb41a receptor has substantially the same amino acid sequence as the human fb41a receptor encoded by plasmid FB41a (ATCC Accession No. 209449). In a further embodiment, the mammalian fb41a receptor has substantially the same amino acid sequence as that shown in FIGS. 2A-2C (Seq. I.D. No. 2). In another embodiment, the mammalian fb41a receptor has the amino acid sequence shown in FIGS. 2A-2C (Seq. I.D. No. 2).

In one embodiment, the cell is an insect cell. In another embodiment, the cell is a mammalian cell. In a further embodiment, the cell is nonneuronal in origin. In another embodiment, the nonneuronal cell is a COS-7 cell, 293 human embryonic kidney cell, a CHO cell, a NIH-3T3 cell, a mouse Y1 cell, or a LM(tk−) cell. In one embodiment, the compound is not previously known to bind to a mammalian fb41a receptor.

This invention provides a compound identified by the above-described process.

This invention provides a method of screening a plurality of chemical compounds not known to bind to a mammalian fb41a receptor to identify a compound which specifically binds to the mammalian fb41a receptor, which comprises (a) contacting cells transfected with and expressing DNA encoding the mammalian fb41a receptor with a compound known to bind specifically to the mammalian fb41a receptor; (b) contacting the preparation of step (a) with the plurality of compounds not known to bind specifically to the mammalian fb41a receptor, under conditions permitting binding of compounds known to bind the mammalian fb41a receptor; (c) determining whether the binding of the compound known to bind to the mammalian fb41a receptor is reduced in the presence of the compounds within the plurality of compounds, relative to the binding of the compound in the absence of the plurality of compounds; and if so (d) separately determining the binding to the mammalian fb41a receptor of compounds included in the plurality of compounds, so as to thereby identify the compound which specifically binds to the mammalian fb41a receptor.

This invention provides a method of screening a plurality of chemical compounds not known to bind to a mammalian fb41a receptor to identify a compound which specifically binds to the mammalian fb41a receptor, which comprises (a) preparing a cell extract from cells transfected with and expressing DNA encoding the mammalian fb41a receptor, isolating a membrane fraction from the cell extract, contacting the membrane fraction with a compound known to bind specifically to the mammalian fb41a receptor; (b) contacting the preparation of step (a) with the plurality of compounds not known to bind specifically to the mammalian fb41a receptor, under conditions permitting binding of compounds known to bind the mammalian fb41a receptor; (c) determining whether the binding of the compound known to bind to the mammalian fb41a receptor is reduced in the presence of the compounds within the plurality of compounds, relative to the binding of the compound in the absence of the plurality of compounds; and if so (d) separately determining the binding to the mammalian fb41a receptor of compounds included in the plurality of compounds, so as to thereby identify the compound which specifically binds to the mammalian fb41a receptor.

In one embodiment of the above-described methods, the mammalian fb41a receptor is a human fb41a receptor. In another embodiment, the cell is a mammalian cell. In a further embodiment, the mammalian cell is non-neuronal in origin. In another embodiment, the non-neuronal cell is a COS-7 cell, a 293 human embryonic kidney cell, a LM(tk−) cell, a CHO cell, a mouse Y1 cell, or an NIH-3T3 cell.

This invention also provides a method of detecting expression of a mammalian fb41a receptor by detecting the presence of mRNA coding for the mammalian fb41a receptor which comprises obtaining total mRNA from the cell and contacting the mRNA so obtained from a nucleic acid probe under hybridizing conditions, detecting the presence of mRNA hybridizing to the probe, and thereby detecting the expression of the mammalian fb41a receptor by the cell.

This invention further provides a method of detecting the presence of a mammalian fb41a receptor on the surface of a cell which comprises contacting the cell with an antibody under conditions permitting binding of the antibody to the receptor, detecting the presence of the antibody bound to the cell, and thereby detecting the presence of the mammalian fb41a receptor on the surface of the cell.

This invention provides a method of determining the physiological effects of varying levels of activity of mammalian fb41a receptors which comprises producing a transgenic, nonhuman mammal whose levels of mammalian fb41a receptor activity are varied by use of an inducible promoter which regulates mammalian fb41a receptor expression.

This invention also provides a method of determining the physiological effects of varying levels of activity of mammalian fb41a receptors which comprises producing a panel of transgenic, nonhuman mammals each expressing a different amount of mammalian fb41a receptor.

This invention provides a method for identifying an antagonist capable of alleviating an abnormality wherein the abnormality is alleviated by decreasing the activity of a mammalian fb41a receptor comprising administering a compound to a transgenic, nonhuman mammal, and determining whether the compound alleviates the physical and behavioral abnormalities displayed by the transgenic, nonhuman mammal as a result of overactivity of a mammalian fb41a receptor, the alleviation of the abnormality identifying the compound as an antagonist. This invention also provides an antagonist identified by the above-described method. This invention further provides a pharmaceutical composition comprising an antagonist identified by the above-described method and a pharmaceutically acceptable carrier. This invention provides a method of treating an abnormality in a subject wherein the abnormality is alleviated by decreasing the activity of a mammalian fb41a receptor which comprises administering to the subject an effective amount of this pharmaceutical composition, thereby treating the abnormality.

This invention provides a method for identifying an agonist capable of alleviating an abnormality in a subject wherein the abnormality is alleviated by increasing the activity of a mammalian fb41a receptor comprising administering a compound to transgenic, nonhuman mammal, and determining whether the compound alleviates the physical and behavioral abnormalities displayed by the transgenic, nonhuman mammal, the alleviation of the abnormality identifying the compound as an agonist. This invention also provides an agonist identified by the above-described method. This invention further provides a pharmaceutical composition comprising an agonist identified by the above-described method and a pharmaceutically acceptable carrier. This invention further provides a method of treating an abnormality in a subject wherein the abnormality is alleviated by increasing the activity of a mammalian fb41a receptor which comprises administering to the subject an effective amount of this pharmaceutical composition, thereby treating the abnormality.

This invention provides a method for diagnosing a predisposition to a disorder associated with the activity of a specific mammalian allele which comprises: (a) obtaining DNA of subjects suffering from the disorder; (b) performing a restriction digest of the DNA with a panel of restriction enzymes; (c) electrophoretically separating the resulting DNA fragments on a sizing gel; (d) contacting the resulting gel with a nucleic acid probe capable of specifically hybridizing with a unique sequence included within the sequence of a nucleic acid molecule encoding a mammalian fb41a receptor and labeled with a detectable marker; (e) detecting labeled bands which have hybridized to the DNA encoding a mammalian fb41a receptor labeled with a detectable marker to create a unique band pattern specific to the DNA of subjects suffering from the disorder; (f) preparing DNA obtained for diagnosis by steps (a)-(e); and (g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same. In one embodiment, a disorder associated with the activity of a specific mammalian allele is diagnosed.

This invention provides a method of preparing the purified mammalian fb41a receptor which comprises: (a) inducing cells to express the mammalian fb41a receptor; (b) recovering the mammalian fb41a receptor from the induced cells; and (c) purifying the mammalian fb41a receptor so recovered.

This invention provides a method of preparing the purified mammalian fb41a receptor which comprises: (a) inserting nucleic acid encoding the mammalian fb41a receptor in a suitable vector; (b) introducing the resulting vector in a suitable host cell; (c) placing the resulting cell in suitable condition permitting the production of the isolated mammalian fb41a receptor; (d) recovering the mammalian fb41a receptor produced by the resulting cell; and (e) purifying the mammalian fb41a receptor so recovered.

This invention provides a process for determining whether a chemical compound is a mammalian fb41a receptor agonist which comprises contacting cells transfected with and expressing DNA encoding the mammalian fb41a receptor with the compound under conditions permitting the activation of the mammalian fb41a receptor, and detecting an increase in mammalian fb41a receptor activity, so as to thereby determine whether the compound is a mammalian fb41a receptor agonist. This invention also provides a process for determining whether a chemical compound is a mammalian fb41a receptor antagonist which comprises contacting cells transfected with and expressing DNA encoding the mammalian fb41a receptor with the compound in the presence of a known mammalian fb41a receptor agonist, under conditions permitting the activation of the mammalian fb41a receptor, and detecting a decrease in mammalian fb41a receptor activity, so as to thereby determine whether the compound is a mammalian fb41a receptor antagonist. In one embodiment, the mammalian fb41a receptor is a human fb41a receptor.

This invention further provides a pharmaceutical composition which comprises an amount of a mammalian fb41a receptor agonist determined by the above-described process effective to increase activity of a mammalian fb41a receptor and a pharmaceutically acceptable carrier. In one embodiment, the mammalian fb41a receptor agonist is not previously known.

This invention provides a pharmaceutical composition which comprises an amount of a mammalian fb41a receptor antagonist determined by the above-described process effective to reduce activity of a mammalian fb41a receptor and a pharmaceutically acceptable carrier. In one embodiment, the mammalian fb41a receptor antagonist is not previously known.

This invention provides a process for determining whether a chemical compound specifically binds to and activates a mammalian fb41a receptor, which comprises contacting cells producing a second messenger response and expressing on their cell surface the mammalian fb41a receptor, wherein such cells do not normally express the mammalian fb41a receptor, with the chemical compound under conditions suitable for activation of the mammalian fb41a receptor, and measuring the second messenger response in the presence and in the absence of the chemical compound, a change in the second messenger response in the presence of the chemical compound indicating that the compound activates the mammalian fb41a receptor. In one embodiment, the second messenger response comprises chloride channel activation and the change in second messenger is an increase in the level of inward chloride current.

This invention also provides a process for determining whether a chemical compound specifically binds to and inhibits activation of a mammalian fb41a receptor, which comprises separately contacting cells producing a second messenger response and expressing on their cell surface the mammalian fb41a receptor, wherein such cells do not normally express the mammalian fb41a receptor, with both the chemical compound and a second chemical compound known to activate the mammalian fb41a receptor, and with only the second chemical compound, under conditions suitable for activation of the mammalian fb41a receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian fb41a receptor. In one embodiment, the second messenger response comprises chloride channel activation and the change in second messenger response is a smaller increase in the level of inward chloride current in the presence of both the chemical compound and the second chemical compound than in the presence of only the second chemical compound.

In one embodiment of the above-described processes, the mammalian fb41a receptor is a human fb41a receptor. In another embodiment, the human fb41a receptor has substantially the same amino acid sequence as encoded by the plasmid FB41a (ATCC Accession No. 209449). In a further embodiment, the human fb41a receptor has substantially the same amino acid sequence as that shown in FIGS. 2A-2C (Seq. I.D. No. 2). In another embodiment, the human fb41a receptor has an amino acid sequence identical to the amino acid sequence shown in FIGS. 2A-2C (Seq. I.D. No. 2). In an embodiment, the cell is an insect cell. In a further embodiment, the cell is a mammalian cell. In a still further embodiment, the mammalian cell is nonneuronal in origin. In another embodiment, the nonneuronal cell is a COS-7 cell, CHO cell, 293 human embryonic kidney cell, NIH-3T3 cell or LM(tk−) cell. In an embodiment, the compound is not previously known to bind to a mammalian fb41a receptor. This invention also provides a compound determined by the above-described processes.

This invention also provides a pharmaceutical composition which comprises an amount of a mammalian fb41a receptor agonist determined by the above-described processes effective to increase activity of a mammalian fb41a receptor and a pharmaceutically acceptable carrier. In one embodiment, the mammalian fb41a receptor agonist is not previously known.

This invention further provides a pharmaceutical composition which comprises an amount of a mammalian fb41a receptor antagonist determined by the above-described processes effective to reduce activity of a mammalian fb41a receptor and a pharmaceutically acceptable carrier. In one embodiment, the mammalian fb41a receptor antagonist is not previously known.

This invention provides a method of screening a plurality of chemical compounds not known to activate a mammalian fb41a receptor to identify a compound which activates the mammalian fb41a receptor which comprises: (a) contacting cells transfected with and expressing the mammalian fb41a receptor with the plurality of compounds not known to activate the mammalian fb41a receptor, under conditions permitting activation of the mammalian fb41a receptor; (b) determining whether the activity of the mammalian fb41a receptor is increased in the presence of the compounds; and if so (c) separately determining whether the activation of the mammalian fb41a receptor is increased by each compound included in the plurality of compounds, so as to thereby identify the compound which activates the mammalian fb41a receptor. In one embodiment, the mammalian fb41a receptor is a human fb41a receptor.

This invention provides a method of screening a plurality of chemical compounds not known to inhibit the activation of a mammalian fb41a receptor to identify a compound which inhibits the activation of the mammalian fb41a receptor, which comprises: (a) contacting cells transfected with and expressing the mammalian fb41a receptor with the plurality of compounds in the presence of a known mammalian fb41a receptor agonist, under conditions permitting activation of the mammalian fb41a receptor; (b) determining whether the activation of the mammalian fb41a receptor is reduced in the presence of the plurality of compounds, relative to the activation of the mammalian fb41a receptor in the absence of the plurality of compounds; and if so (c) separately determining the inhibition of activation of the mammalian fb41a receptor for each compound included in the plurality of compounds, so as to thereby identify the compound which inhibits the activation of the mammalian fb41a receptor. In one embodiment, the mammalian fb41a receptor is a human fb41a receptor.

In one embodiment of the above-described methods, the cell is a mammalian cell. In another embodiment, the mammalian cell is non-neuronal in origin. In a further embodiment, the non-neuronal cell is a COS-7 cell, a 293 human embryonic kidney cell, a LM(tk−) cell or an NIH-3T3 cell.

This invention provides a pharmaceutical composition comprising a compound identified by the above-described methods effective to increase mammalian fb41a receptor activity and a pharmaceutically acceptable carrier.

This invention also provides a pharmaceutical composition comprising a compound identified by the above-described methods effective to decrease mammalian fb41a receptor activity and a pharmaceutically acceptable carrier.

This invention further provides a method of measuring polypeptide activation in an oocyte expression system such as a Xenopus oocyte expression system or melanophore. In an embodiment, polypeptide activation is determined by measurement of ion channel activity. In another embodiment, polypeptide activation is measured by aequerin luminescence.

Expression of genes in Xenopus oocytes is well known in the art (Coleman, A., 1984; Masu, Y.,et al., 1994) and is performed using microinjection of native mRNA or in vitro synthesized mRNA into frog oocytes. The preparation of in vitro synthesized mRNA can be performed by various standard techniques (Sambrook, et al. 1989) including using T7 polymerase with the mCAP RNA mapping kit (Stratagene).

This invention provides a method of treating an abnormality in a subject wherein the abnormality is alleviated by increasing the activity of a mammalian fb41a receptor which comprises administering to the subject an amount of a compound which is a mammalian fb41a receptor agonist effective to treat the abnormality. In separate embodiments, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a visceral innervation disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder, or migraine.

This invention provides a method of treating an abnormality in a subject wherein the abnormality is alleviated by decreasing the activity of a mammalian fb41a receptor which comprises administering to the subject an amount of a compound which is a mammalian fb41a receptor antagonist effective to treat the abnormality. In separate embodiments, the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a visceral innervation disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder or migraine.

This invention also provides the use of mammalian fb41a receptors for analgesia.

This invention will be better understood from the Experimental Details which follow. However, one skilled in the art will readily appreciate that the specific methods and results discussed are merely illustrative of the invention as described more fully in the claims which follow thereafter.

Experimental Details

Materials and methods

Cloning and Sequencing of a Novel Human G Protein-coupled Receptor (fb41a).

A human placental genomic phage library (2.3×10⁶ total recombinants; Stratagene, LaJolla, Calif.) was screened using transmembrane (TM) oligonucleotide probes derived from a new human NPY clone hp25a, later known as human NPY4 (Bard, et al., 1997). Each probe consisted of overlapping oligomers labeled with [³²P]dTP using the Klenow fragment of DNA polymerase. The following oligomers were used:

| | | |
|---|---|---|
| TMI: | | |
| hl428: | 5′-TGATGGTCTTCATCGTCACTTCCTACAGCATTGAGACTGT | (SEQ ID NO. 5) |
| | CGTGGGGGTC-3′ | |
| hl429: | 5′-CAGTCACACACATCAGGCAGAGGTTACCCAGGACCCCCACGA | (SEQ ID NO. 6) |
| | CAGTCTCAA-3′ | |
| TMII: | | |
| hl424: | 5′- ACCTGCTTATCGCCAACCTGGCCTTCTCTGACTTCCTCATG | (SEQ ID NO. 7) |
| | TGCC-3′ | |
| hl425: | 5′-ACGGCGGTCAGCGGCTGGCAGAGGAGGCACATGAGGAAGTCA | (SEQ. ID NO. 8) |
| | GAG-3′ | |
| TMIII: | | |
| hl448: | 5′-CGGAATTCTCCACTCTGGATCATGTATCATGAC-3′ | (SEQ ID NO. 9) |
| hl449: | 5′-GGTCTCTCTGGCCTCTCAGCAAGCTTCAGGGCTTCATGC-3′ | (SEQ ID NO. 10) |
| TMIV: | | |
| hl426: | 5′-GGCCTACCTGGGGATTGTGCTCTGGGTCATTGCCTGTGTC | (SEQ ID NO. 11) |
| | CT-3′ | |
| hl427: | 5′-GCTGTTGGCCAGGAAGGGCAGGGAGAGGACACAGGCAATGAC | (SEQ ID NO. 12) |
| | CCA-3′ | |
| TMV: | | |
| ms450: | 5′-ACCATCTACACCACCTTCCTGCTCCTCTTCCAGTACTGCCTC | (SEQ ID NO. 13) |
| | CCA-3′ | |
| ms451: | 5′-CATAACAGACCAGGATGAAGCCCAGTGGGAGGCAGTACTGGA | (SEQ ID NO. 14) |
| | AGA-3′ | |
| TMVI: | | |
| hl417: | 5′-AATGTGGTGCTGGTGGTGATGGTGGTGGCCTTTGCCGTGCTC | (SEQ ID NO. 15) |
| | TGG-3′ | |
| hl418: | 5′-GGCTGTTGAACCATGCAGAGGCAGCCAGAGCACGGCAAAGG | (SEQ ID NO. 16) |
| | CCA-3′ | |
| TMVII: | | |
| hl419: | 5′-TCATCTTCTTACTGTGCCACTTGCTTGCCATGCCTCCACCTG | (SEQ ID NO. 17) |
| | CG-3′ | |
| hl420: | 5′-AGAAAGCCATAGATGAATGGGTTGACGCAGGTGGAGGCCATG | (SEQ ID NO. 18) |
| | GCA-3′ | |

Hybridization of nitrocellulose filter overlays of the plates was performed at medium stringency: 40° C. in a solution containing 37.5% formamide, 5× SSC (1× SSC in 0.15M sodium chloride, 0.015M sodium citrate), 1X Denhardt's solution (0.02% polyvinylpyrrolindone, 0.02% Ficoll, 0.02% BSA), 7 mM Tris, 7% SDS and 25 μg/ml sonicated salmon sperm DNA. The filters were washed at 45° C. in 0.1× SSC containing 0.5% SDS and exposed at −70° C. to Kodak XAR film in the presence of an

A positive signal on plate 26 was isolated on a tertiary plating and labeled clone igm26a. A 1.6 kb fragment from a PstI digest was identified by southern blot analysis, and subcloned into pUC and used to transform E.coli XL1 cells. Sequencing of the clone was by the Sanger dideoxy method using Sequenase (U.S. Biochemicals Corp.).

A 45-nucleotide oligomer was designed from the NH₂ end of clone igm26a and labeled with ³²P-ATP using polynucleotide kinase. This probe was used to screen a human fetal brain CDNA library (Clonetech) plated out as above. Hybridization of the filter overlays was at high stringency: 40° C. in a solution containing 50% formamide, 5× SSC (1× SSC in 0.15M sodium chloride, 0.015M sodium citrate), 1× Denhardt's solution (0.02% polyvinylpyrrolindone, 0.02% Focoll, 0.02% BSA), 7 mM Tris, 7% SDS and 25 μg/ml sonicated salmon sperm DNA. The filters were washed at 50° C. in 0.1× SSC containing 0.5% SDS and exposed at −70° C. to Kodak XAR film in the presence of an intensifying screen.

A positive signal on plate 41 was isolated on a tertiary plating and labeled clone fb41a. Both a 0.8 kb fragment and a 0.7 kb fragment from an EcoRI digest were identified by southern blot analysis. The fragments were subcloned into separate pUC vectors and used to transform E.coli XL1 cells. Both preparations were sequenced as described above.

The 0.7 kb subclone was digested with EcoRI and SpeI yielding a new 0.6 kb fragment. To obtain full-length clone, this new fragment was subcloned together with the 0.8 kb EcoRI fragment into the expression vector pEXJ. DNA was prepared from this new construct of fb41a, called JB719, and was sequenced on both strands.

Isolation of a Fragment of the Rat Homoloque of JB719

To obtain a fragment of the rat homologue of JB719, rat genomic DNA (Clonetech) was amplified with a forward PCR primer corresponding to TMI of JB719 (BB559) and a reverse primer corresponding to TMIII of JB719 (BB560). PCR was performed with the Expand Long Template PCR System (Boeringer Mannheim) under the following conditions: 30 sec at 94° C., 1.5 min at 50° C., 1.5 min at 68° C. for 40 cycles, with a pre- and post-incubation of 5 min at 94° C. and 7 min at 68° C., respectively. A 300 base pair band was isolated, subcloned using the TA cloning kit (Invitrogen), and sequenced using the AVI BigDye cycle sequencing protocol (Perkin Elmer). The sequence was run and analyzed on an ABI PRISM 377 BigDye Terminator Cycle Sequencing Kit Sequencer. Forward and reverse PCR primers (BB575, also incorporating an EcoRI restriction site, and BB576, also incorporating a BamHI restriction site) were designed against this sequence and used to amplify a band from rat genomic DNA using the following conditions: 30 sec at 94° C., 1.5 min at 68° C. for 35 cycles, with a pre- and post-incubation of 5 min at 94° C. and 5 min at 68° C., respectively. The PCR product was digested with EcoRI and BamHI, and a 259 base-pair fragment was gel-purified and ligated into pGEM. Miniprep cultures were prepared for two transformants, fb41a-1a and fb41a-1b, and both were sequenced as above. Fb41a-1a was renamed pGEM-rfb41a-p.

Primers used:

| | | |
|---|---|---|
| BB559: | 5′-GCCAAGATTGTCATTGGGATGGC-3′ | (SEQ. ID NO. 19) |
| BB560: | 5′-CTGTCAATGGCGATGGCCAGCAG-3′ | (SEQ. ID NO. 20) |
| BB575: | 5′-AGTACTGAATTCTTTGGTGGGCATCATGCTGGTGTG-3′ | (SEQ. ID NO. 21) |
| BB576: | 5′-ATGTCAGGATCCGGCGTTAGTGGACACGTAGAGGGAG-3′ | (SEQ. ID NO. 22) |

The plasmid pGEM-rfb41a-p was deposited on Nov. 11, 1998, with the American Type Culture Collection (ATCC), 10801 University Blvd., Manassas, Va., U.S.A. under the provisions of the Budapest Treaty for the International Recognition of the Deposit of Microorganisms for the Purposes of Patent Procedure and was accorded ATCC Accession No. 203460.

Isolation of the Full-length Rat fb41a Receptor Gene

Fb41a-pGEM-r is a partial rat fb41a clone. It is anticipated that a molecular biologist skilled in the art may isolate the full-length version of the rat fb41a receptor gene using standard molecular biology techniques and approaches such as those briefly described below:

Approach #1: One could use primers designed against the rat fb41a fragment sequence to screen in-house rat cDNA plasmid libraries. Alternatively, one could use a ³²P-labeled oligonucleotide probe designed against the rat fb41a fragment sequence to screen commercial rat phage cDNA libraries.

Approach #2: Standard molecular biology techniques may be used to screen commercial rat genomic libraries, either cosmid or phage, with a ³²P-labeled oligonucleotide probe designed against the rat fb41a fragment sequence. Using this approach one would obtain the sequence for the entire coding region of rat fb41a receptor as well as any introns contained within this region. One could then design a forward primer 5′ of the initiating methionine and a reverse primer 3′ of the stop codon. These primers could then be used to amplify a full-length intronless rat fb41a gene, using rat cDNA as the target template.

Approach #3: As yet another alternative method, one could utilize 5′ RACE and 3′ RACE to determine the additional sequences of the rat fb41a receptor. 5′ RACE could be performed on rat cDNA using a reverse primer derived from known sequence of the rat fb41a fragment, and 3′ RACE could be performed on rat cDNA using a forward primer derived from known sequence of the rat fb41a fragment. These RACE products would be sequenced to determine the full sequence of the rat fb41a receptor. One could then design a forward primer 5′ of the initiating methionine and a reverse primer 3′ of the stop codon. These primers could then be sued to amplify a full-length intronless rat fb41a gene, using rat cDNA as the target template.

Cell Culture

COS-7 cells are grown on 150 mm plates in DMEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of COS-7 cells are trypsinized and split 1:6 every 3-4 days.

Human embryonic kidney 293 cells are grown on 150 mm plates in DMEM with supplements (10% bovine calf serum, 4 mM glutamine, 100 units/ml penicillin/100 μg/ml streptomycin) at 37° C., 5% CO₂. Stock plates of 293 cells are trypsinized and split 1:6 every 3-4 days.

Mouse fibroblast LM(tk−) cells are grown on 150 mm plates in D-MEM with supplements (Dulbecco's Modified Eagle Medium with 10% bovine calf serum, 4 mM glutamine, 100 units/ml penicillin/100 μg/ml streptomycin) at 37° C., 5% CO₂. Stock plates of LM(tk−) cells are trypsinized and split 1:10 every 3-4 days.

Chinese hamster ovary (CHO) cells were grown on 150 mm plates in HAM's F-12 medium with supplements (10% bovine calf serum, 4 mM L-glutamine and 100 units/ml penicillin/ 100 ug/ml streptomycin) at 37° C., 5% CO₂. Stock plates of CHO cells are trypsinized and split 1:8 every 3-4 days.

Mouse embryonic fibroblast NIH-3T3 cells are grown on 150 mm plates in Dulbecco's Modified Eagle Medium (DMEM) with supplements (10% bovine calf serum, 4 mM glutamine, 100 units/ml penicillin/100 μg/ml streptomycin) at 37° C., 5% CO₂. Stock plates of NIH-3T3 cells are trypsinized and split 1:15 every 3-4 days.

Sf9 and Sf21 cells are grown in monolayers on 150 mm tissue culture dishes in TMN-FH media supplemented with 10% fetal calf serum, at 27° C., no CO₂. High Five insect cells are grown on 150 mm tissue culture dishes in Ex-Cell 400™ medium supplemented with L-Glutamine, also at 27° C., no CO₂.

Transient Transfection

Receptors studied may be transiently transfected into COS-7 cells by the DEAE-dextran method using 1 μg of DNA/10⁶ cells (Cullen, 1987). In addition, Schneider 2 Drosophila cells may be cotransfected with vectors containing the receptor gene under control of a promoter which is active in insect cells, and a selectable resistance gene, eg., the G418 resistant neomycin gene, for expression of the polypeptides disclosed herein.

Stable Transfection

DNA encoding the human receptor disclosed herein may be co-transfected with a G-418 resistant gene into the human embryonic kidney 293 cell line by a calcium phosphate transfection method (Cullen, 1987). Stably transfected cells are selected with G-418.

Membrane Preparations

LM(tk−) cells stably transfected with the DNA encoding the human receptor disclosed herein may be routinely converted from an adherent monolayer to a viable suspension. Adherent cells are harvested with trypsin at the point of confluence, resuspended in a minimal volume of complete DMEM for a cell count, and further diluted to a concentration of 10⁶ cells/ml in suspension media (10% bovine calf serum, 10% lOX Medium 199 (Gibco), 9 mM NaHCO₃, 25 mM glucose, 2 mM L-glutamine, 100 units/ml penicillin/100 μg/ml streptomycin, and 0.05% methyl cellulose). Cell suspensions are maintained in a shaking incubator at 37° C., 5% CO₂ for 24 hours. Membranes harvested from cells grown in this manner may be stored as large, uniform batches in liquid nitrogen. Alternatively, cells may be returned to adherent cell culture in complete DMEM by distribution into 96-well microtiter plates coated with poly-D-lysine (0.01 mg/ml) followed by incubation at 37° C., 5% C02 for 24 hours.

Generation of Baculovirus

The coding region of DNA encoding the human receptor disclosed herein may be subcloned into pBlueBacIII into existing restriction sites or sites engineered into sequences 5′ and 3′ to the coding region of the polypeptides. To generate baculovirus, 0.5 μg of viral DNA (BaculoGold) and 3 μg of DNA construct encoding a polypeptide may be co-transfected into 2×10⁶ Spodoptera frugiperda insect Sf9 cells by the calcium phosphate co-precipitation method, as outlined in by Pharmingen (in Baculovirus Expression Vector System: Procedures and Methods Manual). The cells then are incubated for 5 days at 27° C.

The supernatant of the co-transfection plate may be collected by centrifugation and the recombinant virus plaque purified. The procedure to infect cells with virus, to prepare stocks of virus and to titer the virus stocks are as described in Pharmingen's manual.

Radioligand Binding Assays

Cells may be screened for the presence of endogenous human receptor using radioligand binding or functional assays (described in detail in the following experimental description). Cells with either no or a low level of the endogenous human receptor disclosed herein present may be transfected with the human receptor.

Transfected cells from culture flasks are scraped into 5 ml of Tris-HCl, 5 mM EDTA, pH 7.5, and lysed by sonication. The cell lysates are centrifuged at 1000 rpm for 5 min. at 4° C., and the supernatant is centrifuged at 30,000× g for 20 min. at 4° C. The pellet is suspended in binding buffer (50 mM Tris-HCl, 5 mM MgSO₄, 1 mM EDTA at pH 7.5 supplemented with 0.1% BSA, 2 μg/ml aprotinin, 0.5 mg/ml leupeptin, and 10 μg/ml phosphoramidon). Optimal membrane suspension dilutions, defined as the protein concentration required to bind less than 10% of the added radioligand, are added to 96-well polpropylene microtiter plates containing ³H-labeled compound, unlabeled compounds, and binding buffer to a final volume of 250 μl. In equilibrium saturation binding assays membrane preparations are incubated in the presence of increasing concentrations of [³H]-labeled compound. The binding affinities of the different compounds are determined in equilibrium competition binding assays, using [³H]-labeled compound in the presence of ten to twelve different concentrations of the displacing ligands. Binding reaction mixtures are incubated for 1 hr at 30° C., and the reaction stopped by filtration through GF/B filters treated with 0.5% polyethyleneimine, using a cell harvester. Radioactivity may be measured by scintillation counting and data are analyzed by a computerized non-linear regression program. Non-specific binding is defined as the amount of radioactivity remaining after incubation of membrane protein in the presence of unlabeled. Protein concentration may be measured by the Bradford method using Bio-Rad Reagent, with bovine serum albumin as a standard.

Functional Assays

Cells may be screened for the presence of endogenous mammalian receptor using radioligand binding or functional assays (described in detail in the above or following experimental description, respectively). Cells with no or a low level of endogenous receptor present may be transfected with the mammalian receptor for use in the following functional assays.

A wide spectrum of assays can be employed to screen for the presence of orphan receptor ligands. These range from traditional measurements of phosphatidyl inositol, cAMP, Ca⁺⁺, and K⁺, for example; to systems measuring these same second messengers but which have been modified or adapted to be higher throughput, more generic, and more sensitive; to cell based platforms reporting more general cellular events resulting from receptor activation such as metabolic changes, differentiation, and cell division/proliferation, for example; to high level organism assays which monitor complex physiological or behavioral changes thought to be involved with receptor activation including cardiovascular, analgesic, orexigenic, anxiolytic, and sedation effects, for example.

Cyclic AMP (cAMP) Formation Assay

The receptor-mediated inhibition of cyclic AMP (cAMP) formation may be assayed in transfected cells expressing the mammalian receptors. Cells are plated in 96-well plates and incubated in Dulbecco's phosphate buffered saline (PBS) supplemented with 10 mM HEPES, 5 mM theophylline, 2 μg/ml aprotinin, 0.5 mg/ml leupeptin, and 10 μg/ml phosphoramidon for 20 min at 37° C., in 5% CO₂. Test compounds are added and incubated for an additional 10 min at 37° C. The medium is then aspirated and the reaction stopped by the addition of 100 mM HCl. The plates are stored at 4° C. for 15 min, and the cAMP content in the stopping solution measured by radioimmunoassay. Radioactivity may be quantified using a gamma counter equipped with data reduction software.

Arachidonic Acid Release Assay

Cells stably transfected with the mammalian receptor are seeded into 96 well plates and grown for 3 days in HAM's F-12 with supplements. ³H-arachidonic acid (specific activity=0.75 μCi/ml) is delivered as a 100 μL aliquot to each well and samples were incubated at 37° C., 5% CO₂ for 18 hours. The labeled cells are washed three times with 200 μL HAM's F-12. The wells are then filled with medium (200 μL) and the assay is initiated with the addition of peptides or buffer (22 μL). Cells are incubated for 30 min at 37° C., 5% CO₂. Supernatants are transferred to a microtiter plate and evaporated to dryness at 75° C. in a vacuum oven. Samples are then dissolved and resuspended in 25 μL distilled water. Scintillant (300 μL) is added to each well and samples are counted for ³H in a Trilux plate reader. Data are analyzed using nonlinear regression and statistical techniques available in the GraphPAD Prism package (San Diego, Calif.).

Intracellular Calcium Mobilization Assay

The intracellular free calcium concentration may be measured by microspectroflourometry using the fluorescent indicator dye Fura-2/AM (Bush et al, 1991). Stably transfected cells are seeded onto a 35 mm culture dish containing a glass coverslip insert. Cells are washed with HBS and loaded with 100 μL of Fura-2/AM (10 μM) for 20 to 40 min. After washing with HBS to remove the Fura-2/AM solution, cells are equilibrated in HBS for 10 to 20 min. Cells are then visualized under the 40× objective of a Leitz Fluovert FS microscope and fluorescence emission is determined at 510 nM with excitation wavelengths alternating between 340 nM and 380 nM. Raw fluorescence data are converted to calcium concentrations using standard calcium concentration curves and software analysis techniques.

Phosphoinositide Metabolism Assay

Cells stably expressing the mammalian receptor cDNA are plated in 96-well plates and grown to confluence. The day before the assay the growth medium is changed to 100 μl of medium containing 1% serum and 0.5 μCi [³H]myo-inositol, and the plates are incubated overnight in a CO₂ incubator (5% CO₂ at 37° C.). Alternatively, arachidonic acid release may be measured if [³H]arachidonic acid is substituted for the³ [H]myo-inositol. Immediately before the assay, the medium is removed and replaced by 200 μL of PBS containing 10 mM LiCl, and the cells are equilibrated with the new medium for 20 min. During this interval cells are also equilibrated with the antagonist, added as a 10 μL aliquot of a 20-fold concentrated solution in PBS. The [³H]inositol-phosphates accumulation from inositol phospholipid metabolism may be started by adding 10 μL of a solution containing the agonist. To the first well 10 μL may be added to measure basal accumulation, and 11 different concentrations of agonist are assayed in the following 11 wells of each plate row. All assays are performed in duplicate by repeating the same additions in two consecutive plate rows. The plates are incubated in a CO₂ incubator for 1 hr. The reaction may be terminated by adding 15 μL of 50% v/v trichloroacetic acid (TCA), followed by a 40 min. incubation at 4° C. After neutralizing TCA with 40 μL of 1 M Tris, the content of the wells may be transferred to a Multiscreen HV filter plate (Millipore) containing Dowex AG1-X8 (200-400 mesh, formate form). The filter plates are prepared adding 200 μL of Dowex AG1-X8 suspension (50% v/v, water: resin) to each well. The filter plates are placed on a vacuum manifold to wash or elute the resin bed. Each well is washed 2 times with 200 μL of water, followed by 2×200 μL of 5 mM sodium tetraborate/60 mM ammonium formate. The [³H]IPs are eluted into empty 96-well plates with 200 μL of 1.2 M ammonium formate/0.1 formic acid. The content of the wells is added to 3 ml of scintillation cocktail, and the radioactivity is determined by liquid scintillation counting.

GTPγS Functional Assay

Membranes from cells transfected with the mammalian receptors are suspended in assay buffer (50 mM Tris, 100 mM NaCl, 5 mM MgCl₂, pH 7.4) supplemented with 0.1% BSA, 0.1% bacitracin and 10 μM GDP. Membranes are incubated on ice for 20 minutes, transferred to a 96-well Millipore microtiter GF/C filter plate and mixed with GTPγ³⁵S (e.g., 250,000 cpm/sample, specific activity ˜1000 Ci/mmol) plus or minus GTPγS (final concentration=100 μM). Final membrane protein concentration≈90 μg/ml. Samples are incubated in the presence or absence of porcine galanin (final concentration=1 μM) for 30 min. at room temperature, then filtered on a Millipore vacuum manifold and washed three times with cold assay buffer. Samples collected in the filter plate are treated with scintillant and counted for ³⁵S in a Trilux (Wallac) liquid scintillation counter. It is expected that optimal results are obtained when the mammalian receptor membrane preparation is derived from an appropriately engineered heterologous expression system, i.e., an expression system resulting in high levels of expression of the mammalian receptor and/or expressing G-proteins having high turnover rates (for the exchange of GDP for GTP). GTPγS assays are well-known in the art, and it is expected that variations on the method described above, such as are described by e.g., Tian et al. (1994) or Lazareno and Birdsall (1993), may be used by one of ordinary skill in the art.

MAP Kinase Assay

MAP kinase (mitogen activated kinase) may be monitored to evaluate receptor activation. MAP kinase is activated by multiple pathways in the cell. A primary mode of activation involves the ras/raf/MEK/MAP kinase pathway. Growth factor (tyrosine kinase) receptors feed into this pathway via SHC/Grb-2/SOS/ras. Gi coupled receptors are also known to activate ras and subsequently produce an activation of MAP kinase. Receptors that activate phospholipase C (Gq and Gll) produce diacylglycerol (DAG) as a consequence of phosphatidyl inositol hydrolysis. DAG activates protein kinase C which in turn phosphorylates MAP kinase.

MAP kinase activation can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the mitogen and then extracted with Laemmli buffer. The soluble fraction is applied to an SDS-PAGE gel and proteins are transferred electrophoretically to nitrocellulose or Immobilon. Immunoreactive bands are detected by standard Western blotting technique. Visible or chemiluminescent signals are recorded on film and may be quantified by densitometry.

Another approach is based on evaluation of the MAP kinase activity via a phosphorylation assay. Cells are stimulated with the mitogen and a soluble extract is prepared. The extract is incubated at 30° C. for 10 min with gamma-32-ATP, an ATP regenerating system, and a specific substrate for MAP kinase such as phosphorylated heat and acid stable protein regulated by insulin, or PHASI. The reaction is terminated by the addition of H₃PO₄ and samples are transferred to ice. An aliquot is spotted onto Whatman P81 chromatography paper, which retains the phosphorylated protein. The chromatrography paper is washed and counted for ³²P in a liquid scintillation counter. Alternatively, the cell extract is incubated with gamma-32-ATP, an ATP regenerating system, and biotinylated myelin basic protein bound by streptavidin to a filter support. The myelin basic protein is a substrate for activated MAP kinase. The phosphorylation reaction is carried out for 10 min at 30° C. The extract can then by aspirated through the filter, which retains the phosphorylated myelin basic protein. The filter is washed and counted for ³²P by liquid scintillation counting.

Cell Proliferation Assay

Receptor activation of a G protein coupled receptor may lead to a mitogenic or proliferative response which can be monitored via ³H-thymidine uptake. When cultured cells are incubated with ³H-thymidine, the thymidine translocates into the nuclei where it is phosphorylated to thymidine triphosphate. The nucleotide triphosphate is then incorporated into the cellular DNA at a rate that is proportional to the rate of cell growth. Typically, cells are grown in culture for 1-3 days. Cells are forced into quiescence by the removal of serum for 24 hrs. A mitogenic agent is then added to the media. 24 hrs later, the cells are incubated with ³H-thymidine at specific activities ranging from 1 to 10 uCi/ml for 2-6 hrs. Harvesting procedures may involve trypsinization and trapping of cells by filtration over GF/C filters with or without a prior incubation in TCA to extract soluble thymidine. The filters are processed with scintillant and counted for ³H by liquid scintillation counting. Alternatively, adherant cells are fixed in MeOH or TCA, washed in water, and solubilized in 0.05% deoxycholate/0.1 N NaOH. The soluble extract is transferred to scintillation vials and counted for ³H by liquid scintillation counting.

Promiscuous Second Messenger Assays

It is not possible to predict, a priori and based solely upon the GPCR sequence, which of the cell's many different signaling pathways any given orphan receptor will naturally use. It is possible, however, to coax receptors of different functional classes to signal through a pre-selected pathway through the use of promiscuous G_{α} subunits. For example, by providing a cell based receptor assay system with an endogenously supplied promiscuous G_{α} subunit such as_{α16}G or a chimeric G_{α} subunit such as G_{αzq}, a GPCR, which might normally prefer to couple through a specific signaling pathway (e.g., G_{g}, Gᵢ, G_{q}, G₀, etc.), can be made to couple through the pathway defined by the promiscuous G_{α} subunit and upon agonist activation produce the second messenger associated with that subunit's pathway. In the case of G_{α16} and/or G_{αaqz} this would involve activation of the G_{q} pathway and production of the second messenger phosphotidyl inositol. Through the use of similar strategies and tools, it is possible to bias receptor signaling through pathways producing other second messengers such as Ca⁺⁺, cAMP, and ⁺K currents, for example.

It follows that the promiscuous interaction of the exogenously supplied G_{α} subunit with the orphan receptor alleviates the need to carry out a different assay for each possible signaling pathway and increases the chances of detecting a functional signal upon receptor activation.

For phosphotidyl inositol (PI) measurements Cos-7 cells are typically used as the reporter cell. Cos-7 cells are transiently transfected by electroporation (BioRad Gene Pulser II, 0.23 kV, 950 μF, 4.5×10⁶ cells/cuvette) with 5 μg of individual expression vectors containing orphan receptor(s), control receptor(s), and/or promiscuous G_{α} subunits. The transfected cells are then plated into 96-well tissue culture plates (100,000 cells/well in complete DMEM (10% BCS, 1%P/S, 2% Gln)) and incubated at 37° C., 5% CO₂, O/N. To assay for the production of PI, the cells are labeled with [3H]myo-inositol (0.5 μCi/well) in complete DMEM while incubating O/N as before. The next day the [³H] medium is poured off and the cells are washed 1× with PBS. After washing, 90 μl of 10 mM LiCi in PBS/Ca⁺⁺, Mg⁺⁺ is added to each well, and the plates are then incubated for 15 min. at 37° C., 5% CO₂. The cells are then challenged with ligand (defined drugs are presented at 10 μM final concentration) for 30 min. at 37° C., 5% CO₂ O/N. The stimulation is terminated by the addition of 100 μl cold 5% TCA (4° C., at least 10 min.). The plate contents are then transferred to a 96-well filter plate previously packed with a slurry of 50% Dowex AGI0X8 (100 μl/well). The cells are washed 3× with 200 μl 5 mM myoinositol and the [³H]-inositol phosphates are then eluted with 75 μl 1.2M ammonium formate/0.1M formic acid into an empty Wallac 96-well scintillation plate. 200 μl of SuperMix Scintillation cocktail is added to each well, mixed well, allowed to equilibrate and counted in a Micro Beta Trilux scintillation counter.

Microphysiometric Measurement of Orphan Receptor Mediated Extracellular Acidification Rates

Because cellular metabolism is intricately involved in a broad range of cellular events (including receptor activation of multiple messenger pathways), the use of microphysiometric measurements of cell metabolism can in principle provide a generic assay of cellular activity arising from the activation of any orphan receptor regardless of the specifics of the receptor's signaling pathway.

General guidelines for transient receptor expression, cell preparation and microphysiometric recording are described elsewhere (Salon, J. A. and Owicki, J. A., 1996). Orphan receptors and/or control vectors are transiently expressed in CHO-K1 cells, by liposome mediated transfection according to the manufacturers recommendations (LipofectAMINE, GibcoBRL, Gaithersburg, Md.), and maintained in Ham's F-12 complete (10% serum). A total of 10 μg of DNA is used to transfect each 75 cm² flask which had been split 24 hours prior to the transfection and judged to be 70-80% confluent at the time of transfection. 24 hours post transfection, the cells are harvested and 3×10⁵ cells seeded into microphysiometet capsules. Cells are allowed to attach to the capsule membrane for an additional 24 hours; during the last 16 hours, the cells are switched to serum-free F-12 complete to minimize ill-defined metabolic stimulation caused by assorted serum factors. On the day of the experiment the cell capsules are transferred to the microphysiometer and allowed to equilibrate in recording media (low buffer RPMI 1640, no bicarbonate, no serum (Molecular Devices Corporation, Sunnyvale, Calif.) containing 0.1% fatty acid free BSA), during which a baseline measurement of basal metabolic activity is established.

A standard recording protocol specifies a 100 μl/min flow rate, with a 2 min total pump cycle which includes a 30 sec flow interruption during which the acidification rate measurement is taken. Ligand challenges involve a 1 min 20 sec exposure to the sample just prior to the first post challenge rate measurement being taken, followed by two additional pump cycles for a total of 5 min 20 sec sample exposure. Typically, drugs in a primary screen are presented to the cells at 10 μM final concentration. Ligand samples are then washed out and the acidification rates reported are expressed as a percentage increase of the peak response over the baseline rate observed just prior to challenge.

Clearly, an important aspect of understanding orphan receptors is the identification and characterization of their ligands. The scope and structural diversity of activating ligands (agonists) anticipated to be discovered for orphans is represented by the known universe of ligands for the GPCR superfamily. These range from large viral coat proteins and glycoproteins, to peptides, lipids, small molecules, and even activating ions. The diversity can be further expanded upon if we consider the many known synthetic antagonists specific for GPCR subtypes.

Discrete GPCR Ligand Library

Functional assays of orphan receptors include a preliminary test of a small library of compounds containing representative agonists for all known GPCRs as well as other compounds which may be agonists for prospective GPCRs or which may be effectors for targets peripherally involved with GPCRs. The collection currently comprises approximately 180 compounds, (including small molecules, hormones, preprohormones, and peptides, for example), for more than 45 described classes of GPCRs (serotonin, dopamine, noradrenalin, opiods, etc.) And additionally includes ligands for known or suspected but not necessarily pharmacologically characterized or cloned GPCR families. The diversity of the library can be expanded to include agonist and antagonist compounds specific for GPCR subtypes, combinatorial peptide and/or small molecule libraries, natural product collections, and the like. To facilitate robotic handling, the substances are distributed as either separate or pooled compound concentrates in 96 well plates and stored frozen as ready to use reagent plates.

Peptide Transmitter cDNA Library

It is anticipated that a large portion of orphan receptors will have peptide or protein molecules as their natural ligands. Accordingly, approaches employing the expression cloning of novel peptide transmitters using assay systems and cDNA libraries tailored to this task are a viable approach to the problem of identifying orphan receptor ligands.

Isolation of Endogenous Ligands

Due to the limited understanding of the structural basis of transmitter diversity, it is very likely that successful identification of orphan receptor ligands will come not through efforts that rely solely on screening synthetic chemical or peptide libraries, but rather through the screening of ligand rich biological extracts from organisms and tissues that express the receptor itself as well. The logic of this hypothesis is that where nature has evolved a regulatory system based on a novel receptor it must also provide the means to activate the receptor via a novel endogenous transmitter substance. Accordingly, it is important in outlining a strategy to include the orphan receptor based screening of extracts derived from naturally occurring biological sources and the subsequent purification and characterization of any orphan receptor linked biological activity present in said extracts.

A general approach is to screen high resolution HPLC fractions of various tissue extracts for orphan receptor activity, employing one or more cellular based assays as described elsewhere. In general, a receptor based assay system employing reporter cells, which either transiently or stably express a particular orphan receptor(s), will be challenged with HPLC fractions derived from tissues thought to harbor transmitter substances and monitor signal transduction readouts for heterotrimeric G protein activation. To circumvent the problem of endogenous GPCRs (orphan or extaneous) in the reporter lines that may be activated by one or more endogenous transmitters in the extracts, the parent host cell lines (i.e. not heterologously expressing the orphan receptor) will be tested in parallel. Positive hits for orphan receptor linked activity will be evidenced by signaling present in the cell line heterologously expressing the orphan receptor but absent in the parent line. Tissue sources for extraction will be chosen by several criteria, including the localization of the orphan receptor itself, the relative abundance of known transmitter substances, and the potential involvement of the tissue in important disease states. Extraction procedures will depend upon the structural class of ligand being sought after and could include but not be restricted to; neutral aqueous extraction for protein molecules, acid extraction for peptide molecules and small molecule chemical transmitters, and organic solvent extraction for lipid or sterol molecules.

Purification of orphan receptor linked biological activity will depend upon the structural characteristic of the transmitter substance, but could include various low, medium and high pressure chromatographic methods based on size exclusion, anion/cation, hydrophobic, and affinity interaction matrices and could employ either normal or reversed phase conditions. Preparative electrophoresis in one and two dimensions would also, in some circumstances, be a viable approach for purification.

In addition to various signal transduction assays which would be used to track bio-activity during purification, various biophysical methods would be employed to analyze the complexity and structural characteristics of the purified fractions. These methods would include, but not be limited to, UV-vis absorbance spectroscopy, proteolytic fragmentation, mass spectrometry, amino acid sequencing, and ultimately nuclear magnetic resonance spectrometry and/or X-ray crystallographic determination of the purified transmitter molecule's 3-dimensional structure.

Receptor/G Protein Co-transfection Studies

A strategy for determining whether fb41a can couple preferentially to selected G proteins involves co-transfection of fb41a receptor cDNA into a host cell together with the cDNA for a G protein alpha sub-unit. Examples of G alpha sub-units include members of the Gαi/Gαo class (including Gαt2 and Gαz), the Gαq class, the Gαs class, and the Gα12/13 class. A typical procedure involves transient transfection into a host cell such as COS-7. Other host cells may be used. A key consideration is whether the cell has a downstream effector (a particular adenylate cyclase, phospholipase C, or channel isoform, for example) to support a functional response through the G protein under investigation. G protein beta gamma sub-units native to the cell are presumed to complete the G protein heterotrimer; otherwise specific beta and gamma sub-units may be co-transfected as well. Additionally, any individual or combination of alpha, beta, or gamma subunits may be co-transfected to optimize the functional signal mediated by the receptor.

The receptor/G alpha co-transfected cells are evaluated in a binding assay, in which case the radioligand binding may be enhanced by the presence of the optimal G protein coupling or in a functional assay designed to test the receptor/G protein hypothesis. In one example, fb41a may be hypothesized to inhibit cAMP accumulation through coupling with G alpha sub-units of the Gαi/Gαo class. Host cells co-transfected with fb41a and appropriate G alpha sub-unit cDNA are stimulated with forskolin +/− fb41a agonist, as described above in cAMP methods. Intracellular cAMP is extracted for analysis by radioimmunoassay. Other assays may be substituted for cAMP inhibition, including GTPγ³⁵S binding assays and inositol phosphate hydrolysis assays. Host cells transfected with fb41a minus G alpha or with G alpha minus fb41a would be tested simultaneously as negative controls. fb41a receptor expression in transfected cells may be confirmed in ¹²⁵I-fb41a protein binding studies using membranes from transfected cells. G alpha expression in transfected cells may be confirmed by Western blot analysis of membranes from transfected cells, using antibodies specific for the G protein of interest.

The efficiency of the transient transfection procedure is a critical factor for signal to noise in an inhibitory assay, much more so than in a stimulatory assay. If a positive signal present in all cells (such as forskolin-stimulated cAMP accumulation) is inhibited only in the fraction of cells successfully transfected with receptor and G alpha, the signal to noise ratio will be poor. One method for improving the signal to noise ratio is to create a stably transfected cell line in which 100% of the cells express both the receptor and the G alpha subunit. Another method involves transient co-transfection with a third cDNA for a G protein-coupled receptor which positively regulates the signal which is to be inhibited. If the co-transfected cells simultaneously express the stimulatory receptor, the inhibitory receptor, and a requisite G protein for the inhibitory receptor, then a positive signal may be elevated selectively in transfected cells using a receptor-specific agonist. An example involves co-transfection of COS-7 cells with 5-HT4, fb41a, and a G alpha sub-unit. Transfected cells are stimulated with a 5-HT4 agonist +/− fb41a protein. Cyclic AMP is expected to be elevated only in the cells also expressing fb41a and the G alpha subunit of interest, and a fb41a-dependent inhibition may be measured with an improved signal to noise ratio.

It is to be understood that the cell lines described herein are merely illustrative of the methods used to evaluate the binding and function of the mammalian receptors of the present invention, and that other suitable cells may be used in the assays described herein.

Methods for Recording Currents in Xenopus oocytes

Female Xenopus laevis (Xenopus-1, Ann Arbor, Mich.) are anesthetized in 0.2% tricain (3-aminobenzoic acid ethyl ester, Sigma Chemical Corp.) and a portion of ovary is removed using aseptic technique (Quick and Lester, 1994). Oocytes are defolliculated using 2 mg/ml collagenase (Worthington Biochemical Corp., Freehold, N.J.) in a solution containing 87.5 mM NaCl, 2 mM KCl, 2 mM MgCl₂ and 5 mM HEPES, pH 7.5. Oocytes may be injected (Nanoject, Drummond Scientific, Broomall, Pa.) with mammalian mRNA. Other oocytes may be injected with a mixture of mammalian mRNA and mRNA encoding the genes for G-protein-activated inward rectifiers (GIRK1 and GIRK4, U.S. Pat. Nos. 5,734,021 and 5,728,535). Genes encoding G-protein inwardly rectifying K⁺ (GIRK) channels 1 and 4 (GIRK1 and GIRK4) are obtained by PCR using the published sequences (Kubo et al., 1993; Dascal et al., 1993; Krapivinsky et al., 1995 and 1995b) to derive appropriate 5′ and 3′ primers. Human heart cDNA is used as template together with appropriate primers.

In each primer pair, the upstream primer may contain a BamHI site and the downstream primer may contain an EcoRI site to facilitate cloning of the PCR product into pcDNA1-Amp (Invitrogen). The transcription template for the mammalian receptor may be similarly obtained. mRNAs are prepared from separate DNA plasmids containing the complete coding regions of the mammalian receptor, GIRK1, and GIRK4. Plasmids are linearized and transcribed using the T7 polymerase (Message Machine, Ambion). Alternatively, mRNA may be translated from a template generated by PCR, incorporating a T7 promoter and a poly A⁺ tail. Each oocyte receives 2 ng each of GIRK1 and GIRK4 mRNA in combination with 25 ng of mammalian receptor mRNA. After injection of mRNA, oocytes are incubated at 16° on a rotating platform for 3-8 days. Dual electrode voltage clamp (GeneClamp, Axon Instruments Inc., Foster City, Calif.) is performed using 3 M KCl-filled glass microelectrodes having resistances of 1-3 Mohms. Unless otherwise specified, oocytes are voltage clamped at a holding potential of −80 mV. During recordings, oocytes are bathed in continuously flowing (2-5 ml/min) medium containing 96 mM NaCl, 2 mM KC1, 2 mM CaCl₂, 2 mM MgCl₂, and 5 mM HEPES, pH 7.5 (ND96), or, in the case of oocytes expressing GIRK1 and GIRK4, elevated K⁺ containing 96 mM KCl, 2 mM NaCl, 2 mM CaCl₂, 2 mM MgCl₂, and 5 mM HEPES, pH 7.5 (hK). Drugs are applied by switching from a series of gravity fed perfusion lines.

Heterologous expression of GPCRs in Xenopus oocytes has been widely used to determine the identity of signaling pathways activated by agonist stimulation (Gundersen et al., 1983; Takahashi et al., 1987). Activation of the phospholipase C (PLC) pathway is assayed by applying test compound in ND96 solution to oocytes previously injected with mRNA for the mammalian receptor and observing inward currents at a holding potential of −80 mV. The appearance of currents that reverse at −25 mV and display other properties of the Ca⁺⁺-activated Cl⁻ (chloride) channel is indicative of mammalian receptor-activation of PLC and release of IP3 and intracellular Ca⁺⁺. Such activity is exhibited by GPCRs that couple to G_{q}.

Measurement of inwardly rectifying K⁺ (potassium) channel (GIRK) activity is monitored in oocytes that have been co-injected with mRNAs encoding the mammalian receptor, GIRK1, and GIRK4. The two GIRK gene products co-assemble to form a G-protein activated potassium channel known to be activated (i.e., stimulated) by a number of GPCRs that couple to Gᵢ or Gₒ (Kubo et al., 1993; Dascal et al., 1993). Oocytes expressing the mammalian receptor plus the two GIRK subunits are tested for test compound responsivity by measuring K⁺ currents in elevated K⁺ solution (hK). Activation of inwardly rectifying currents that are sensitive to 300 μM Ba⁺⁺ signifies the mammalian receptor coupling to a Gᵢ or Gₒ pathway in the oocytes.

Localization Studies

Development of probes: Using full length cDNA encoding the fb41a receptor as a template, polymerase chain reaction (PCR) was used to amplify a 425 base pair fragment corresponding to nucleotides 705-1130 of the coding sequence. PCR generated fragments were subcloned into the EcoRI and HindIII sites of a plasmid vector pGEM 7zf (Promega Corp.), which contains sp6 and T7 RNA polymerase promoter sites. This construct was linearized with EcoRI and sp6 RNA polymerase was used to synthesize radiolabeled antisense strands of RNA.

A probe coding for human glyceraldehyde 3-phosphate dehydrogenase (GAPDH) gene, a constitutively expressed protein, was used concurrently. GAPDH is expressed at a relatively constant level in most tissue and its detection is used to compare expression levels of the human fb41a gene in different regions.

Development of probes for rat fb41a: Isolation and cloning of cDNA sequences encoding rat fb41a are described elsewhere. Radiolabeled RNA probes for rat fb41a were synthesized in the same manner as those shown for human fb41a.

Synthesis of probes: fb41a and GAPDH cDNA sequences preceded by phage polymerase promoter sequences were used to synthesize radiolabeled riboprobes. Conditions for the synthesis of riboprobes were: 0.25-1.0 μg linearized template, 1.5 μl of ATP, GTP, UTP (10 mM each), 3 μl dithiothreitol (0.1M), 30 units RNAsin RNAse inhibitor, 0.5-1.0 μl (15-20 units/μl) RNA polymerase, 7.0 μl transcription buffer (Promega Corp.), and 12.5 μl α³²P-CTP (specific activity 3,000 Ci/mmol). 0.1 mM CTP (0.02-1.0 μl) was added to the reactions, and the volumes were adjusted to 35 μl with DEPC-treated water. Labeling reactions were incubated at 37° C. for 60 minutes, after which 3 units of RQ1 RNAse-free DNAse (Promega Corp.) were added to digest the template. Riboprobes were separated from unincorporated nucleotides using Microspin S-300 columns (Pharmacia Biotech). TCA precipitation and liquid scintillation spectrometry were used to measure the amount of label incorporated into the probe. A fraction of all riboprobes synthesized was size-fractionated on 0.25 mm thick 7M urea, 4.5% acrylamide sequencing gels. These gels were apposed to screens and the autoradiograph scanned using a phosphorimager (Molecular Dynamics) to confirm that the probes synthesized were full-length and not degraded.

Solution hybridization/ribonuclease protection assay (RPA): For solution hybridization 2.0 μg of mRNA isolated from tissues were used. Negative controls consisted of 30 μg transfer RNA (tRNA) or no tissue blanks. All mRNA samples were placed in 1.5 ml microfuge tubes and vacuum dried. Hybridization buffer (40 μl of 400 mM NaCl, 20 mM Tris, Ph 6.4, 2 mM EDTA, in 80% formamide) containing 0.25-2.0 E⁶ counts of each probe were added to each tube. Samples were heated at 90° C. for 5 min, after which the temperature was lowered to 45 or 55° C. for hybridization. After hybridization for 14-18 hr, the RNA/probe mixtures were digested with RNAse A (Sigma) and RNAse T1 (Life Technologies). A mixture of 2.0 μg RNAse A and 1000 units of RNAse T1 in a buffer containing 330 mM NACl, 10 mM Tris (pH 8.0) and 5 mM EDTA (400 μl) was added to each sample and incubated for 90 min at room temperature. After digestion with RNAses, 20 μl of 10% SDS and 50 μg proteinase K were added to each tube and incubated at 37° C. for 15 min. Samples were extracted with phenol/chloroform:isoamyl alcohol and precipitated in 2 volumes of ethanol for 1 hr at −70° C. Pellet Paint (Novagen) was added to each tube (2.0 μg) as a carrier to facilitate precipitation. Following precipitation, samples were centrifuged, washed with cold 70% ethanol, and vacuum dried. Samples were dissolved in formamide loading buffer and size-fractionated on a urea/acrylamide sequencing gel (7.0 M urea, 4.5% acrylamide in Tris-borate-EDTA) Gels were dried and apposed to storage phosphor screens and scanned using a phosphorimager (Molecular Dynamics, Sunnydale, Calif.).

RT-PCR

For the detection of low levels of RNA encoding fb41a, RT-PCR was carried out on mRNA extracted from human tissue. Reverse transcription and PCR reactions were carried out in 50 μl volumes using EzrTth DNA polymerase (Perkin Elmer). Primers with the following sequences were used:

RA rFB41aF:

| | |
|---|---|
| RA rFB41aF: | |
| GCATCATGCTGGTGTGTGGCATCG | (Seq. I.D. No. 23) |
| RA rFB41aB: | |
| GTTAGTGGACACGTAGAGGGAGACG | (Seq. I.D. No. 24) |

Each reaction contained 0.2 μg mRNA and 0.3 μM of each primer. Concentrations of reagents in each reaction were: 300 μM each of dGTP, DATP, dCTP, dTTP; 2.5 mM Mn(OAc)₂; 50 mM Bicine; 115 mM K acetate, 8% glycerol and 5 units EzrTth DNA polymerase. All reagents for PCR (except mRNA and oligonucleotide primers) were obtained from Perkin Elmer. Reactions were carried out under the following conditions: 65° C., 60 min; 94° C., 2 min; (94° C., 1 min; 65° C., 1 min) 40 cycles, 72° C., 10 min. PCR reactions were size fractionated by agarose gel electrophoresis, DNA stained with ethidium bromide (EtBr) and photographed with UV illumination.

Positive controls for PCR reactions consisted of amplification of the target sequence from a plasmid construct, as well as reverse transcribing and amplifying a known sequence. Negative controls consisted of mRNA blanks as well as primer blanks. To confirm that the mRNA was not contaminated with genomic RNA, samples were digested with RNAses before reverse transcription. Integrity of RNA was assessed by amplification of mRNA coding for GAPDH.

Northern blots and multiple species southern blots: Human multiple tissue northern blots and multiple species southern blots (ZooBlots) were purchased from Clonetech Laboratories, Inc. (Palo Alto, Cslif.). Blots were prehybridized in ExpressHyb hybridization solution (Clonetech Laboratories, Inc.) for one hour at 75° C. After prehybridization, labeled riboprobe (synthesized as previously described for human fb41a) was added (0.5-1.0 E⁶ CPM/ml of hybridization solution). Blots were hybridized overnight at 75° C. After hybridization blots were washed 4 times in washes of increasing stringency. Final wash for all blots was: 0.1× SSC, 75° C. (20× SSC=3M NaCl, 0.3M Na₃ citrate, pH 7.0). After washing, blots were apposed to storage phosphor screens and scanned using a phosphorimager after exposure times of 1-5 days (Molecular Dynamics, Sunnyvale, Calif.)

Results and Discussion

A human genomic placenta library was screened, under reduced stringency conditions, with oligonucleotide probes directed to the seven transmembrane regions of the human Y4 receptor (Bard, et al., 1995). A positively-hybridizing clone was isolated, plaque-purified and characterized by Southern blot analysis and sequencing. The sequence of this clone was used to design a 45 nucleotide oligonucleotide probe which was used to screen a human fetal brain cDNA library. Two fragments from one positively.hybridizing clone, fb41a, were subcloned together into the expression vector pEXJ.

The largest open reading frame in this construct, JB719, contains 1167 nucleotides (FIGS. 1A-1B), which is predicted to encode a protein of 389 amino acids (FIGS. 2A-2B). A second potential initiating methionine is present and would predict a protein of 386 amino acids. Hydropathy analysis of the protein is consistent with a putative topography of seven transmembrane domains, indicative of the G protein-coupled receptor family (FIGS. 2A-2B).

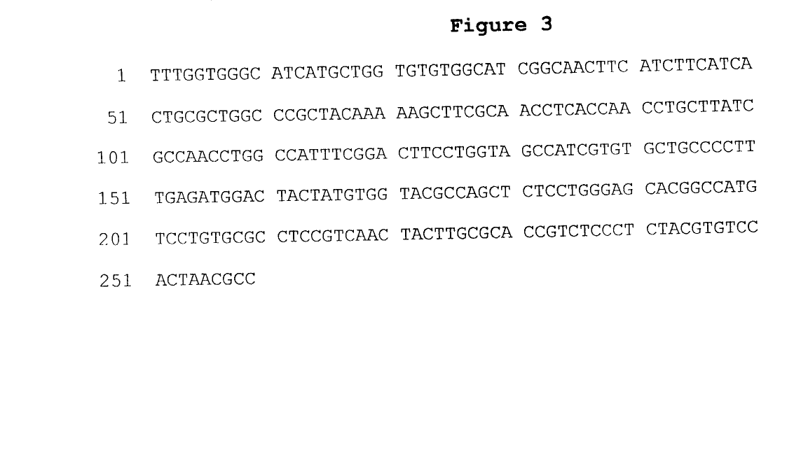

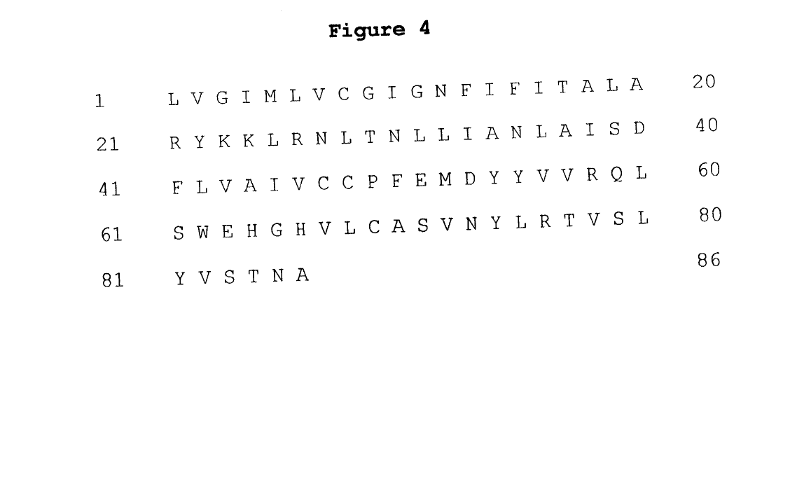

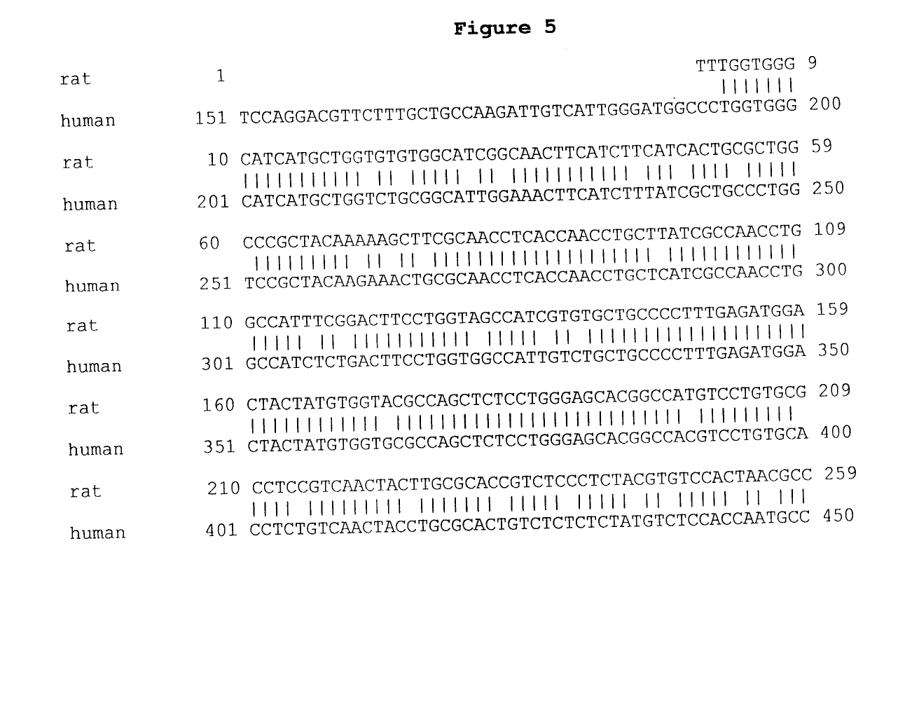

A 300 base pair fragment containing TMs I to III of the rat homologue of JB719 was isolated from rat genomic DNA using primers directed against the human clone. The sequence of this fragment was then used to design primers specific to the rat homologue. Using these primers, a 259 base pair fragment was isolated from rat genomic DNA and subcloned into pGEM (FIGS. 3, 4). The sequence of two clones from independent PCR reactions, fb41a-1a and fb41a-1b, were identical and showed 88% nucleotide identity with JB719 (FIG. 5).

Localization

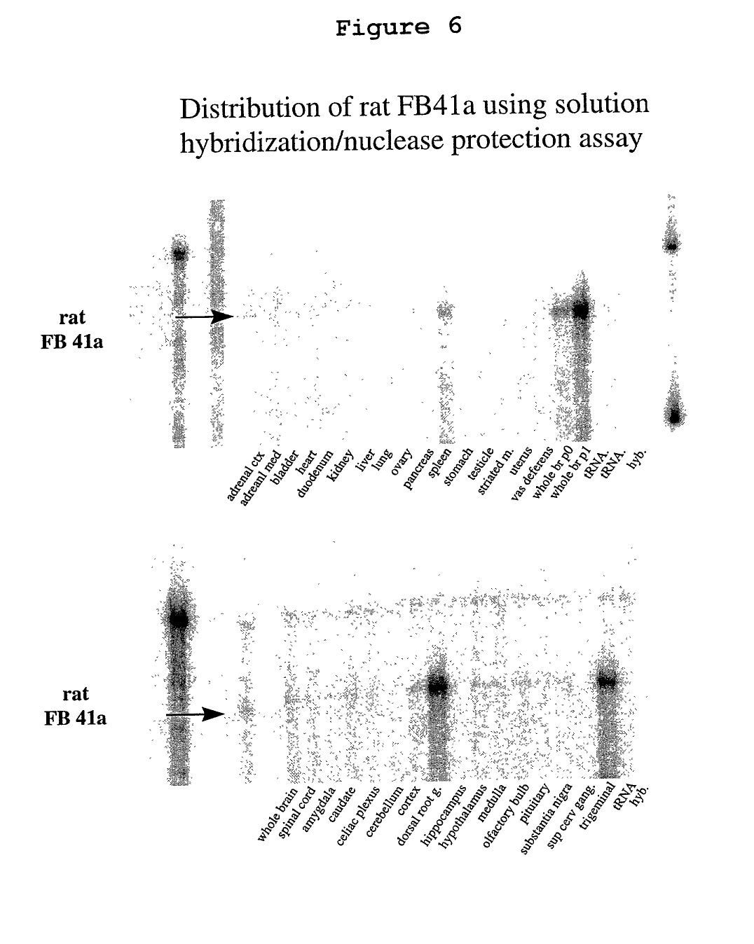

Detection of mRNA coding for rat fb41a: mRNA was isolated from multiple tissues (Table 1) and assayed as described. P0 and P1 indicate post natal days 0 and 1. The distribution of mRNA encoding rat fb41a is widespread with the highest levels found in trigeminal ganglia, dorsal root ganglia and neonatal brains. Lower amounts are found broadly distributed throughout the central nervous system, as well as in peripheral organs (Table 1, FIG. 6). There is good correlation between distribution determined by RT-PCR and RPA. RT-PCR detected rat fb41a in more areas than RPA as it is a more sensitive technique.

High levels of mRNA encoding fb41a in the dorsal root ganglia and trigeminal ganglia with relatively low expression in most of the other regions assayed provides insights for the potential function of fb41a. Primary sensory neurons are located in both dorsal root and trigeminal ganglia. This distribution strongly implicates fb41a as a potential modulator of pain and/or sensory transmission.

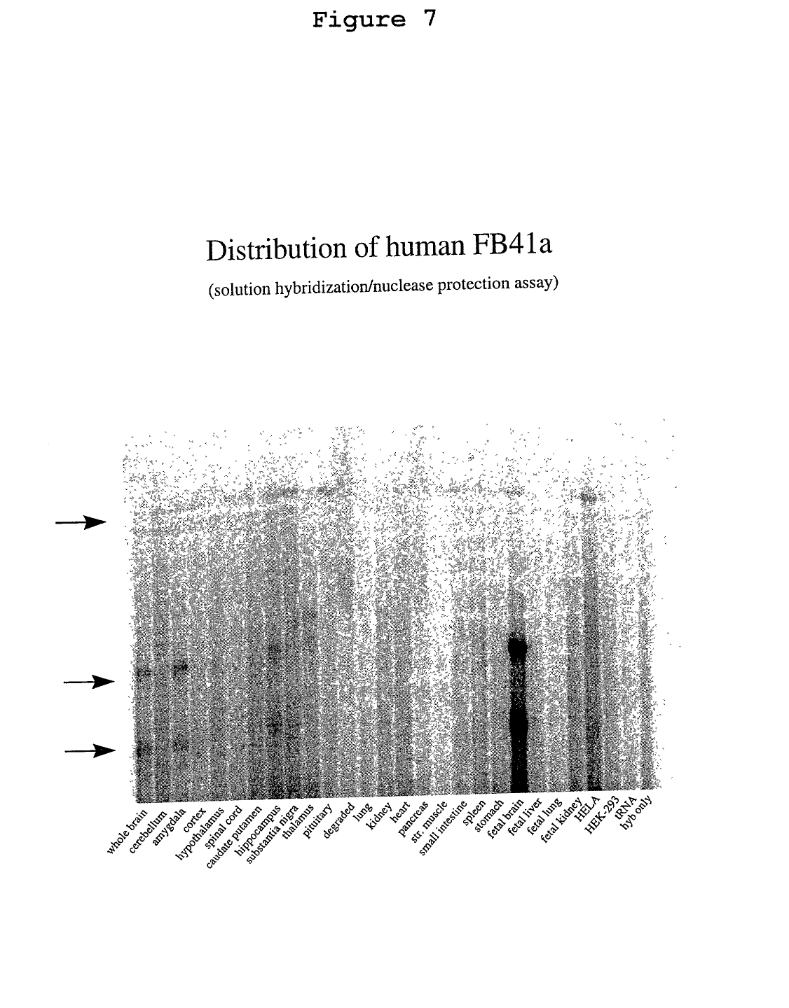

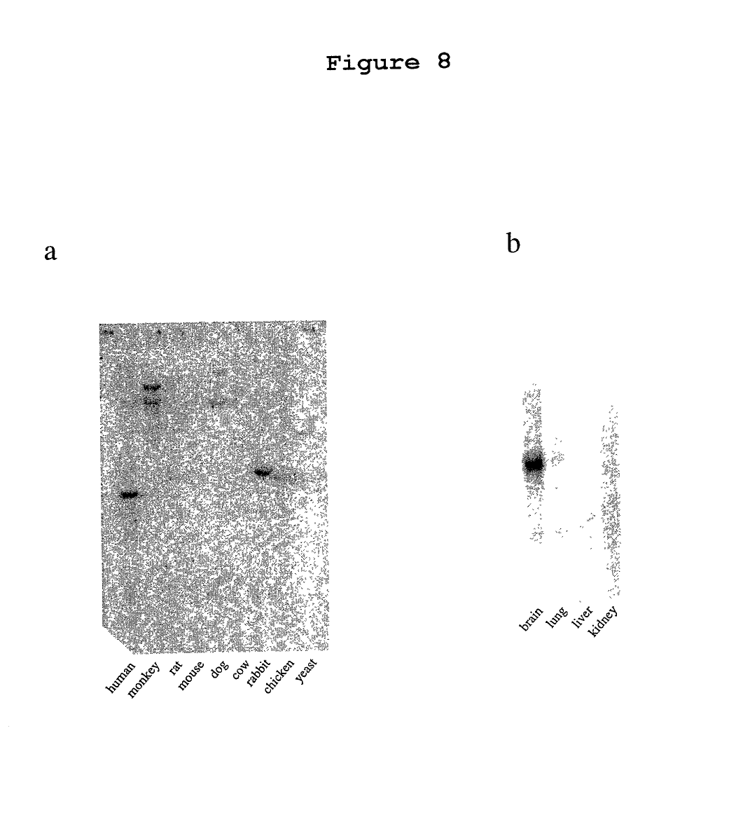

Detection of mRNA coding for human fb41a: mRNA was isolated and assayed as described from areas listed in Table 2. The distribution of mRNA encoding human fb41a is widespread with the highest levels found in fetal brain (25 week gestational age was the only age assayed). Other areas containing mRNA encoding fb41a include the cerebellum and pituitary (FIG. 7). Northern blot analysis of mRNA extracted from fetal brain, fetal lung, fetal liver, and fetal kidney demonstrates a high level of expression in fetal brain, with no detectable specific hybridization in mRNA from the other tissues (FIG. 8B).

Presence of fb41a-like genes in other species: Hybridization of a radiolabeled human fb41a riboprobe to genomic DNA from multiple species on a ZooBlot demonstrates that fb41a-like gene sequences are present in multiple species including human, monkey, rat, dog, cow, rabbit, and yeast (FIG. 8A). This suggests that fb41a is a well conserved receptor that may play a functional role across phylogeny.

TABLE 1
Distribution of mRNA coding for rat fb41a receptor

| | rat | rat | |
|---|---|---|---|
| | fb41a | fb41a | |
| Region | RT-PCR | RPA | Potential Applications |
| adrenal | ++ | − | Regulation of steroid hormones |
| cortex | | | |
| adrenal | ++ | − | Regulation of epinephrine release |
| medulla | | | |
| urinary | − | − | Urinary incontinence |
| bladder | | | |
| duodenum | + | − | Gastrointestinal disorders |
| heart | +/− | − | Cardiovascular indications |
| kidney | ++ | +/− | Electrolyte balance, hypertension |
| liver | ++ | +/− | Diabetes |
| lung | ++ | +/− | Respiratory disorders, asthma |
| ovary | + | +/− | Reproductive function |
| Pancreas | +/− | − | Diabetes, endocrine disorders |
| Spleen | +++ | ++ | Immune disorders |
| stomach | +/− | − | Gastrointestinal disorders |
| striated | +/− | − | Musculoskeletal disorders |
| muscle | | | |
| testicle | + | +/− | Reproductive function |
| Uterus | ++ | − | Reproductive function |
| vas | ++ | − | Reproductive function |
| deferens | | | |
| Whole | + | + | |
| brain | | | |
| Spinal | ++ | +/− | Analgesia, sensory modulation and |
| cord | | | transmission |
| amygdala | ++ | + | |
| caudate/ | + | + | Modulation of dopaminergic function |
| putamen | | | |
| cellac | + | + | modulation of visceral innervation |
| plexus | | | |
| cerebellum | + | − | Motor coordination |
| Cerebral | + | + | Sensory and motor integration, |
| cortex | | | cognition |
| Dorsal | +++ | ++++ | Sensory transmission |
| root | | | |
| ganglia | | | |
| Hippo- | − | − | Cognition/memory |
| campus | | | |
| Hypo- | ++ | + | Appetite/obesity, neuroendocrine |
| thalamus | | | regulation |
| Medulla | + | + | Analgesia, motor coordination |
| Olfactory | + | + | Olfaction |
| bulb | | | |
| Pituitary | + | + | Endocrine/neuroendocrine regulation |
| Substantia | ++ | + | Modulation of dopaminergic function |
| nigra | | | |
| Superior | + | − | Modulation of sympathetic |
| cervical | | | innervation |
| ganglion | | | |
| Trigeminal | +++ | ++++ | Migraine, analgesia, sensory |
| ganglion | | | transmission |
| Whole | NA | ++ | |
| brain P0 | | | |
| Whole | NA | +++ | |
| brain P1 | | | |

NA = not assayed

TABLE 2
Distribution of mRNA coding for human fb41a receptors

| | Human | |
|---|---|---|
| Region | fb41a | Potential applications |
| Liver | + | Diabetes |
| Kidney | +/− | Hypertension, Electrolye balance |
| Lung | +/− | Respiratory disorders, asthma |
| Heart | +/− | cardiovascular indications |
| Small intestine | +/− | Gastrointestinal disorders |
| Striated muscle | +/− | Musculoskeletal disorders |
| Pituitary | ++ | Endocrine/neuroendocrine |
| | | regulation |
| Whole brain | + | |
| Amygdala | + | |
| Cerebral cortex | +/− | Sensory and motor integration, |
| | | cognition |
| Hippocampus | + | Cognition/memory |
| Hypothalamus | ++ | Appetite/obesity, neuroendocrine |
| | | regulation |
| spinal cord | +/− | Analgesia, sensory modulation and |
| | | transmission |
| Cerebellum | ++ | Motor coordination |
| Thalamus | +/− | Sensory integration |
| Substantia nigra | ++ | Modulation of dopaminergic |
| | | function, modulation of motor |
| | | coordination |
| caudate/putamen | + | modulation of dopaminergic |
| | | function |
| fetal brain | ++++ | |
| fetal lung | +/− | |
| fetal kidney | +/− | |
| fetal liver | +/− | |

A comparison of nucleotide and peptide sequences of clone JB719 with sequences contained in the Genbank/EMBL databases reveals that the clone is most related to GPR10 (49% nucleotide identity, 27% amino acid identity), human neurokinin-1 receptor (48% nucleotide identity, 23% amino acid identity), human NPY/PYY/PP Y4 receptor (46% nucleotide identity, 24% amino acid identity), human NPY/PYY/PP Y2 receptor (46% nucleotide identity, 26% amino acid identity), human neurokinin-2 receptor (44% nucleotide identity, 24% amino acid identity) and human orexin-2 receptor (43% nucleotide identity, 23% amino acid identity). In addition a human cosmid clone (Genbank accession number HSE122E9) was identified which contains the first 475 nucleotides of JB719. This region of the cosmid entry was unannotated. The similar level of identity of JB719 to GPCRs of multiple subfamilies (biogenic amine and neuropeptide) indicates that the endogenous ligand could be from any class of molecules interacting with GPCRs. However, it is not yet possible to accurately predict the nature of the endogenous ligand from primary sequence alone. The cloning of the gene encoding fb41a has nevertheless provided the means to explore its physiological roles by pharmacological characterization, and by Northern and in situ mapping of its mRNA distribution. Further, the availability of the DNA encoding the fb41a receptor will facilitate the development of antibodies and antisense technologies useful in defining the functions of the gene product in vivo. Antisense oligonucleotides which target mRNA molecules to selectively block translation of the gene product in vivo have been used successfully to relate the expression of a single gene with its functional sequelae. The cloning of fb41a will allow the use of this approach to explore the functional consequences of blocking the expression of its mRNA without knowledge of its endogenous ligand. Thus, the cloning of this receptor gene provides the means to explore its physiological roles in the nervous system and elsewhere, and may thereby help to elucidate structure/function relationships within the GPCR superfamily.

In conclusion, the primary structure of the protein encoded by the fb41a receptor gene and its lack of close identity with existing GPCRs indicate that the endogenous ligand may represent any class of neuroregulatory substances, and further suggest that additional members of this new receptor subfamily may exist.

### REFERENCES

U.S. Pat. No. 5,602,024 (Gerald et al. Feb. 11, 1997).

Bard J. A., Walker M. W., Branchek T. A. and Weinshank R. L. Cloning and functionl expression of a human Y4 subtype receptor for pancreatic polypeptide, neuropeptide Y, and peptide YY. J. Biol. Chem.270:26762-26765 (1995).

Bradford, M. M. A rapid and sensitive method for the quantitation of microgram quantities of protein utilizing the principle of protein-dye binding. Anal Biochem2:248-54 (1976).

Burns, C. C., Moser, M., Banks, J., Alderete, J. P., and Overbaugh, J. Identification and deletion of sequences required for feline leukemia virus RNA packaging and construction of a high-titer feline leukemia virus packaging cell line. Virology 222:1 14-20 (1996).

Bush, A. B. Borden, L. A. Greene, L. A. and Maxfield, F. R. Nerve growth factor potentiates bradykinin-induced calcium influx and release in PC12 cells. J. Neurochem 57:562-574(1991).

Chu, Y. Y., Tu, K. H., Lee, Y. C., Kuo, Z. J., Lai, H. L., and Chern, Y. Characterization of the rat A2a adenosine receptor gene.DNA Cell Biol 4: 329-37 (1996).

Coleman, A. (1984) Transcription and Translation: A Practical Approach (B. D. Hanes, S. J. Higgins, eds., pp 271-302, IRL Press, Oxford, 1984).

Cullen, B. Use of eukaryotic expression technology in the functional analysis of cloned genes. Methods Enzymol.152:685-704 (1987).

Dascal, N., Schreibmayer, W., Lim, N. F., Wang, W., Chavkin, C., DiMagno, L., Labarca, C., Kieffer, B. L., Gaveriaux-Ruff, C., Trollinger, D., Lester, H. A.,

Davidson, N. (1993) Proc. Natl. Acad. Sci. USA 90:10235-10239.

Fargin, A.; Raymond, J. R.; Lohse, M. J.; Kobilka, B. K.; Caron, M. G.; Lefkowitz, R. J. Nature 335:358-360 (1988).

Fong, T. M.; Huang, R. C.; Yu, H.; Swain, C. J.; Underwood, D.; Cascieri, M. A.; Strader, C. D. Can. J. Physiol. Pharmacol. 73(7):860-865 (July 1995).

Graziano, M. P.; Hey, P. J.; Strader, C.D. Receptors Channels 4(1):9-17 (1996).

Guam, X. M.; Amend, A.; Strader, C. D.; Mol. Pharmacol. 48(3):492-498 (Sep 1995).

Gundersen, C. B., Miledi, R., and Parker, I. Serotonin receptors induced by exogenous messenger RNA in Xenopus oocytes. Proc R Soc Lond B Biol Sci 219:1214 103-9 (1983).

Krapivinsky, G., Gordon, E. A., Wickman B., Velimirovic, B., Krapivinsky, L., Clapham, D. E. (1995) Nature 374:135-141.

Krapivinsky, G., Krapivinsky, L., Velimirovic, B., Wickman, K., Navarro, B., Clapham, D. E., (1995b) J. Biol. Chem. 270:28777-28779.

Kubo, Y., Reuveny, E., Slesinger, P. A., Jan, Y. N., Jan, L.Y. (1993) Nature 364:802-806.

Lazareno, S. and Birdsall N.J.M.Pharmacological characterization of acetylcholine stimulated [35S]-GTPgS binding mediated by human muscarinic ml-m4 receptors: antagonist studies. Br. J. Pharmacology, 109: 1120-1127 (1993)

Masu, Y. et al. (1994) Nature 329:21583-21586.

Miller, J., Germain, R.N., Efficient cell surface expression of class II MHC molecules in the absence of associated invariant chain. J.Exp.Med. 164:1478-1489 (1986).

Quick, M. W. and Lester, H. A. Methods for expression of excitability proteins in Xenopus oocytes. Meth. Neurosci. 19:261-279 (1994).

Salon, J. A. and Owicki, J. C. Real-time measurements of receptor activity: Applications of microphysiometric techniques to receptor biology. In: Methods in Neuroscience Vol. 25:201-223 Academic Press, (1995)

Sambrook, J., Fritsch, E. F., and Maniatis, T., In: Molecular Cloning: A Laboratory Manual, 2nd Edition (Cold Spring Harbor Laboratory, Cold Spring Harbor, N.Y.), 1989.

Southern, E. M. Detection of specific sequences among DNA fragments separated by gel electrophoresis. J. Mol. Biol. 98:503-517 (1975).

Spurney, R. F.; Coffman, T. M. J. Pharmacol. Exp. Ther. 283(1):207-215 (October 1997).

Takahashi, T., Neher, E., and Sakmann, B. Rat brain serotonin receptors in Xenopus oocytes are coupled by intracellular calcium to endogenous channels. Proc Natl Acad Sci U S A 84:14 5063-7 (1987)

Tian, W., Duzic, E., Lanier, S., and Deth R.Determinants of alpha-Adrenergic Receptor Activation of G protein: Evidence for a Precoupled Receptor/G protein State. Molecular Pharmacology, 45: 524-53.(1994).

Underwood, D. J., Strader, C. D., Rivero, R., Patchett, A. A., Greenlee ,W., and Prendergast K. Structural model of antagonist and agonist binding to the angiotensin II, AT1 subtype, G protein coupled receptor. Chem Biol 4:211-21 (1994)

Weinshank, R. L.; Zgombick, J. M.; Macchi, M. J.; Branchek, T. A.; Hartig, P. R. Proc. Natl. Acad. Sci. U.S.A. 89(8):3630-3634 (1992).

What is claimed is:

1. An isolated nucleic acid encoding a mammalian fb41a receptor.

2. The nucleic acid of claim 1, wherein the nucleic acid is DNA.

3. The DNA of claim 2, wherein the DNA is cDNA.

4. The DNA of claim 2, wherein the DNA is genomic DNA.

5. The nucleic acid of claim 1, wherein the nucleic acid is RNA.

6. The nucleic acid of claim 1, wherein the mammalian fb41a receptor is a human fb41a receptor.

7. The nucleic acid of claim 6, wherein the nucleic acid encodes a human fb41a receptor which has an amino acid sequence identical to that encoded by the plasmid FB41a (ATCC Accession No. 209449).

8. An isolated nucleic acid encoding a human fb41a receptor analog.

9. The nucleic acid of claim 6, wherein the human fb41a receptor has an amino acid sequence comprising the amino acid sequence shown in FIGS. 2A-2C (Seq. I.D. No. 2) begining with the methionine at amino acid position 4.

10. A purified mammalian fb41a receptor protein.

11. The purified mammalian fb41a receptor protein of claim 10, wherein the fb41a receptor protein is a human fb41a receptor protein.

12. A vector comprising the nucleic acid of claim 1.

13. A vector comprising the nucleic acid of claim 6.

14. A vector of claim 12 adapted for expression in a bacterial cell which comprises the regulatory elements necessary for expression of the nucleic acid in the bacterial cell operatively linked to the nucleic acid encoding the mammalian fb41a receptor as to permit expression thereof.

15. A vector of claim 12 adapted for expression in an amphibian cell-which comprises the regulatory elements necessary for expression of the nucleic acid in the amphibian cell operatively linked to the nucleic acid encoding the mammalian fb41a receptor as to permit expression thereof.

16. A vector of claim 12 adapted for expression in a yeast cell which comprises the regulatory elements necessary for expression of the nucleic acid in the yeast cell operatively linked to the nucleic acid encoding the mammalian fb41a receptor so as to permit expression thereof.

17. A vector of claim 12 adapted for expression in an insect cell which comprises the regulatory elements necessary for expression of the nucleic acid in the insect cell operatively linked to the nucleic acid encoding the mammalian fb41a receptor so as to permit expression thereof.

18. The vector of claim 17 which is a baculovirus.

19. A vector of claim 12 adapted for expression in a mammalian cell which comprises the regulatory elements necessary for expression of the nucleic acid in the mammalian cell operatively linked to the nucleic acid encoding the mammalian fb41a receptor so as to permit expression thereof.

20. The vector of claim 12, wherein the vector is a plasmid.

21. The plasmid of claim 20 designated FB41a (ATCC Accession No. 209449).

22. A cell comprising the vector of claim 12.

23. A cell of claim 22, wherein the cell is a non-mammalian cell.

24. A cell of claim 23, wherein the non-mammalian cell is a Xenopus oocyte cell or a Xenopus melanophore cell.

25. A cell of claim 22, wherein the cell is a mammalian cell.

26. A mammalian cell of claim 25, wherein the cell is a COS-7 cell, a 293 human embryonic kidney cell, a NIH-3T3 cell, a LM(tk−) cell, a mouse Y1 cell, or a CHO cell.

27. An insect cell comprising the vector of claim 17.

28. An insect cell of claim 27, wherein the insect cell is an Sf9 cell, an Sf21 cell or a HighFive cell.

29. A membrane preparation isolated from the cell of any one of claims 22, 23, 25, or 27.

30. A nucleic acid probe comprising at least 15 nucleotides, which probe specifically hybridizes with a nucleic acid encoding a mammalian fb41a receptor, wherein the probe has a unique sequence corresponding to a sequence present within one of the two strands of the nucleic acid encoding the mammalian fb41a receptor and are contained in plasmid FB41a (ATCC Accession No. 209449).

31. A nucleic acid probe comprising at least 15 nucleotides, which probe specifically hybridizes with a nucleic acid encoding a mammalian fb41a receptor, wherein the probe has a unique sequence corresponding to a sequence present within (a) the nucleic acid sequence shown in FIG. 1A-lB (Seq. I.D. No. 1) or (b) the reverse complement thereto.

32. The nucleic acid probe of claim 30 or 31, wherein the nucleic acid is DNA.

33. The nucleic acid probe of claim 30 or 31, wherein the nucleic acid is RNA.

34. A nucleic acid probe comprising a nucleic acid molecule of at least 15 nucleotides which is complementary to a unique fragment of the sequence of a nucleic acid molecule encoding a mammalian fb41a receptor.

35. A nucleic acid probe comprising a nucleic acid molecule of at least 15 nucleotides which is complementary to the antisense sequence of a unique fragment of the sequence of a nucleic acid molecule encoding a mammalian fb41a receptor.

36. An antisense oligonucleotide having a sequence capable of specifically hybridizing to the RNA of claim 5, so as to prevent translation of the RNA.

37. An antisense oligonucleotide having a sequence capable of specifically hybridizing to the genomic DNA of claim 4.

38. An antisense oligonucleotide of claim 36 or 37, wherein the oligonucleotide comprises chemically modified nucleotides or nucleotide analogues.

39. An antibody capable of binding to a mammalian fb41a receptor encoded by the nucleic acid of claim 1.

40. An antibody of claim 39, wherein the mammalian fb41a receptor is a human fb41a receptor.

41. An agent capable of competitively inhibiting the binding of the antibody of claim 39 to a mammalian fb41a receptor.

42. An antibody of claim 39, wherein the antibody is a monoclonal antibody or antisera.

43. A pharmaceutical composition comprising (a) an amount of the oligonucleotide of claim 36 capable of passing through a cell membrane and effective to reduce expression of a mammalian fb41a receptor and (b)a pharmaceutically acceptable carrier capable of passing through the cell membrane.

44. A pharmaceutical composition of claim 43, wherein the oligonucleotide is coupled to a substance which inactivates mRNA.

45. A pharmaceutical composition of claim 44, wherein the substance which inactivates mRNA is a ribozyme.

46. A pharmaceutical composition of claim 43, wherein the pharmaceutically acceptable carrier comprises a structure which binds to a mammalian fb41a receptor on a cell capable of being taken up by the cells after binding to the structure.

47. A pharmaceutical composition of claim 46, wherein the pharmaceutically acceptable carrier is capable of binding to a mammalian fb41a receptor which is specific for a selected cell type.

48. A pharmaceutical composition which comprises an amount of the antibody of claim 39 effective to block binding of a ligand to a human fb41a receptor and a pharmaceutically acceptable carrier.

49. A transgenic, nonhuman mammal expressing DNA encoding a mammalian fb41a receptor of claim 1.

50. A transgenic, nonhuman mammal comprising a homologous recombination knockout of the native mammalian fb41a receptor.

51. A transgenic, nonhuman mammal whose genome comprises antisense DNA complementary to the DNA encoding a mammalian fb41a receptor of claim 1 so placed within the genome as to be transcribed into antisense mRNA which is complementary to mRNA encoding the mammalian fb41a receptor and which hybridizes to mRNA encoding the mammalian fb41a receptor, thereby reducing its translation.

52. The transgenic, nonhuman mammal of claim 49 or 50, wherein the DNA encoding the mammalian fb41a receptor additionally comprises an inducible promoter.

53. The transgenic, nonhuman mammal of claim 49 or 50, wherein the DNA encoding the mammalian fb41a receptor additionally comprises tissue specific regulatory elements.

54. A transgenic, nonhuman mammal of claim 49, 50, or 51, wherein the transgenic, nonhuman mammal is a mouse.

55. A process for identifying a chemical compound which specifically binds to a mammalian fb41a receptor which comprises contacting cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor.

56. A process for identifying a chemical compound which specifically binds to a mammalian fb41a receptor which comprises contacting a membrane fragment from a cell extract of cells containing DNA encoding and expressing on their cell surface the mammalian fb41a receptor, wherein such cells do not normally express the mammalian fb41a receptor, with the compound under conditions suitable for binding, and detecting specific binding of the chemical compound to the mammalian fb41a receptor.

57. The process of claim 55 or 56, wherein the mammalian fb41a receptor is a human fb41a receptor.

58. The process of claim 55 or 56, wherein the mammalian fb41a receptor has substantially the same amino acid sequence as the mammalian fb41a receptor encoded by plasmid FB41a (ATCC Accession No. 209449).

59. The process of claim 55 or 56, wherein the mammalian fb41a receptor has substantially the same amino acid sequence as that shown in FIGS. 2A-2C (Seq. I.D. No. 2).

60. The process of claim 55 or 56, wherein the mammalian fb41a receptor has the amino acid sequence shown in FIGS. 2A-2C (Seq. I.D. No. 2).

61. The process of claim 59, wherein the compound is not previously known to bind to a mammalian fb41a receptor.

62. A compound identified by the process of claim 61.

63. A process of claim 59, wherein the cell is an insect cell.

64. The process of claim 59, wherein the cell is a mammalian cell.

65. The process of claim 64, wherein the cell is nonneuronal in origin.

66. The process of claim 65, wherein the nonneuronal cell is a COS-7 cell, 293 human embryonic kidney cell, a CHO cell, a NIH-3T3 cell, a mouse Y1 cell, or a LM(tk−) cell.

67. A process of claim 64, wherein the compound is a compound not previously known to bind to a mammalian fb41a receptor.

68. A compound identified by the process of claim 67.

69. A process involving competitive binding for identifying a chemical compound which specifically binds to a mammalian fb41a receptor which comprises separately contacting cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

70. A process involving competitive binding for identifying a chemical compound which specifically binds to a mammalian fb41a receptor which comprises separately contacting a membrane fraction from a cell extract of cells expressing on their cell surface the mammalian fb41a receptor, wherein such cells do not normally express the mammalian fb41a receptor, with both the chemical compound and a second chemical compound known to bind to the receptor, and with only the second chemical compound, under conditions suitable for binding of both compounds, and detecting specific binding of the chemical compound to the mammalian fb41a receptor, a decrease in the binding of the second chemical compound to the mammalian fb41a receptor in the presence of the chemical compound indicating that the chemical compound binds to the mammalian fb41a receptor.

71. A process of claim 69 or 70, wherein the mammalian fb41a receptor is a human fb41a receptor.

72. The process of claim 71, wherein the human fb41a receptor has substantially the same amino acid sequence as the human fb41a receptor encoded by plasmid FB41a (ATCC Accession No. 209449).

73. The process of claim 69 or 70, wherein the mammalian fb41a receptor has substantially the same amino acid sequence as that shown in FIGS. 2A-2C (Seq. I.D. No. 2).

74. The process of claim 69 or 70, wherein the mammalian fb41a receptor has the amino acid sequence shown in FIGS. 2A-2C (Seq. I.D. No. 2).

75. The process of claim 73, wherein the cell is an insect cell.

76. The process of claim 73, wherein the cell is a mammalian cell.

77. The process of claim 76, wherein the cell is nonneuronal in origin.

78. The process of claim 77, wherein the nonneuronal cell is a COS-7 cell, 293 human embryonic kidney cell, a CHO cell, a NIH-3T3 cell, a mouse Y1 cell, or a LM(tk−) cell.

79. The process of claim 78, wherein the compound is not previously known to bind to a mammalian fb41a receptor.

80. A compound identified by the process of claim 79.

81. A method of screening a plurality of chemical compounds not known to bind to a mammalian fb41a receptor to identify a compound which specifically binds to the mammalian fb41a receptor, which comprises
(a) contacting cells transfected with and expressing DNA encoding the mammalian fb41a receptor with a compound known to bind specifically to the mammalian fb41a receptor;
(b) contacting the preparation of step (a) with the plurality of compounds not known to bind specifically to the mammalian fb41a receptor, under conditions permitting binding of compounds known to bind the mammalian fb41a receptor;
(c) determining whether the binding of the compound known to bind to the mammalian fb41a receptor is reduced in the presence of the compounds within the plurality of compounds, relative to the binding of the compound in the absence of the plurality of compounds; and if so
(d) separately determining the binding to the mammalian fb41a receptor of compounds included in the plurality of compounds, so as to thereby identify the compound which specifically binds to the mammalian fb41a receptor.

82. A method of screening a plurality of chemical compounds not known to bind to a mammalian fb41a receptor to identify a compound which specifically binds to the mammalian fb41a receptor, which comprises
(a) preparing a cell extract from cells transfected with and expressing DNA encoding the mammalian fb41a receptor, isolating a membrane fraction from the cell extract, contacting the membrane fraction with a compound known to bind specifically to the mammalian fb41a receptor;
(b) contacting the preparation of step (a) with the plurality of compounds not known to bind specifically to the mammalian fb41a receptor, under conditions permitting binding of compounds known to bind the mammalian fb41a receptor;
(c) determining whether the binding of the compound known to bind to the mammalian fb41a receptor is reduced in the presence of the compounds within the plurality of compounds, relative to the binding of the compound in the absence of the plurality of compounds; and if so
(d) separately determining the binding to the mammalian fb41a receptor of compounds included in the plurality of compounds, so as to thereby identify the compound which specifically binds to the mammalian fb41a receptor.

83. A method of claim 81 or 82, wherein the mammalian fb41a receptor is a human fb41a receptor.

84. A method of claim 81 or 82, wherein the cell is a mammalian cell.

85. A method of claim 84, wherein the mammalian cell is non-neuronal in origin.

86. The method of claim 85, wherein the non-neuronal cell is a COS-7 cell, a 293 human embryonic kidney cell, a LM(tk−) cell, a CHO cell, a mouse Y1 cell, or an NIH-3T3 cell.

87. A method of detecting expression of a mammalian fb41a receptor by detecting the presence of mRNA coding for the mammalian fb41a receptor which comprises obtaining total mRNA from the cell and contacting the mRNA so obtained with the nucleic acid probe of claim 30 under hybridizing conditions, detecting the presence of mRNA hybridizing to the probe, and thereby detecting the expression of the mammalian fb41a receptor by the cell.

88. A method of detecting the presence of a mammalian fb41a receptor on the surface of a cell which comprises contacting the cell with the antibody of claim 39 under conditions permitting binding of the antibody to the receptor, detecting the presence of the antibody bound to the cell, and thereby detecting the presence of the mammalian fb41a receptor on the surface of the cell.

89. A method of determining the physiological effects of varying levels of activity of mammalian fb41a receptors which comprises producing a transgenic, nonhuman mammal of claim 52 whose levels of mammalian fb41a receptor activity are varied by use of an inducible promoter which regulates mammalian fb41a receptor expression.

90. A method of determining the physiological effects of varying levels of activity of mammalian fb41a receptors which comprises producing a panel of transgenic, nonhuman mammals of claim 52 each expressing a different amount of mammalian fb41a receptor.

91. A method for identifying an antagonist capable of alleviating an abnormality wherein the abnormality is alleviated by decreasing the activity of a mammalian fb41a receptor comprising administering a compound to the transgenic, nonhuman mammal of claim 49, 52, 53, or 54, and determining whether the compound alleviates the physical and behavioral abnormalities displayed by the transgenic, nonhuman mammal as a result of overactivity of a mammalian fb41a receptor, the alleviation of the abnormality identifying the compound as an antagonist.

92. An antagonist identified by the method of claim 91.

93. A pharmaceutical composition comprising an antagonist identified by the method of claim 92 and a pharmaceutically acceptable carrier.

94. A method of treating an abnormality in a subject wherein the abnormality is alleviated by decreasing the activity of a mammalian fb41a receptor which comprises administering to the subject an effective amount of the pharmaceutical composition of claim 93, thereby treating the abnormality.

95. A method for identifying an agonist capable of alleviating an abnormality in a subject wherein the abnormality is alleviated by increasing the activity of a mammalian fb41a receptor comprising administering a compound to the transgenic, nonhuman mammal of claim 49, 52, 53, or 54, and determining whether the compound alleviates the physical and behavioral abnormalities displayed by the transgenic, nonhuman mammal, the alleviation of the abnormality identifying the compound as an agonist.

96. An agonist identified by the method of claim 95.

97. A pharmaceutical composition comprising an agonist identified by the method of claim 95 and a pharmaceutically acceptable carrier.

98. A method of treating an abnormality in a subject wherein the abnormality is alleviated by increasing the activity of a mammalian fb41a receptor which comprises administering to the subject an effective amount of the pharmaceutical composition of claim 97, thereby treating the abnormality.

99. A method for diagnosing a predisposition to a disorder associated with the activity of a specific mammalian allele which comprises:
(a) obtaining DNA of subjects suffering from the disorder;
(b) performing a restriction digest of the DNA with a panel of restriction enzymes;
(c) electrophoretically separating the resulting DNA fragments on a sizing gel;
(d) contacting the resulting gel with a nucleic acid probe capable of specifically hybridizing with a unique sequence included within the sequence of a nucleic acid molecule encoding a mammalian fb41a receptor and labeled with a detectable marker;
(e) detecting labeled bands which have hybridized to the DNA encoding a mammalian fb41a receptor of claim 1 labeled with a detectable marker to create a unique band pattern specific to the DNA of subjects suffering from the disorder;
(f) preparing DNA obtained for diagnosis by steps (a)-(e); and
(g) comparing the unique band pattern specific to the DNA of subjects suffering from the disorder from step (e) and the DNA obtained for diagnosis from step (f) to determine whether the patterns are the same or different and to diagnose thereby predisposition to the disorder if the patterns are the same.

100. The method of claim 99, wherein a disorder associated with the activity of a specific mammalian allele is diagnosed.

101. A method of preparing the purified mammalian fb41a receptor of claim 11 which comprises:
(a) inducing cells to express the mammalian fb41a receptor;
(b) recovering the mammalian fb41a receptor from the induced cells; and
(c) purifying the mammalian fb41a receptor so recovered.

102. A method of preparing the purified mammalian fb41a receptor of claim 11 which comprises:
(a) inserting nucleic acid encoding the mammalian fb41a receptor in a suitable vector;
(b) introducing the resulting vector in a suitable host cell;
(c) placing the resulting cell in suitable condition permitting the production of the isolated mammalian fb41a receptor;
(d) recovering the mammalian fb41a receptor produced by the resulting cell; and
(e) purifying the mammalian fb41a receptor so recovered.

103. A process for determining whether a chemical compound is a mammalian fb41a receptor agonist which comprises contacting cells transfected with and expressing DNA encoding the mammalian fb41a receptor with the compound under conditions permitting the activation of the mammalian fb41a receptor, and detecting an increase in mammalian fb41a receptor activity, so as to thereby determine whether the compound is a mammalian fb41a receptor agonist.

104. A process for determining whether a chemical compound is a mammalian fb41a receptor antagonist which comprises contacting cells transfected with and expressing DNA encoding the mammalian fb41a receptor with the compound in the presence of a known mammalian fb41a receptor agonist, under conditions permitting the activation of the mammalian fb41a receptor, and detecting a decrease in mammalian fb41a receptor activity, so as to thereby determine whether the compound is a mammalian fb41a receptor antagonist.

105. A process of claim 103 or 104, wherein the mammalian fb41a receptor is a human fb41a receptor.

106. A pharmaceutical composition which comprises an amount of a mammalian fb41a receptor agonist determined by the process of claim 103 effective to increase activity of a mammalian fb41a receptor and a pharmaceutically acceptable carrier.

107. A pharmaceutical composition of claim 106, wherein the mammalian fb41a receptor agonist is not previously known.

108. A pharmaceutical composition which comprises an amount of a mammalian fb41a receptor antagonist determined by the process of claim 104 effective to reduce activity of a mammalian fb41a receptor and a pharmaceutically acceptable carrier.

109. A pharmaceutical composition of claim 108, wherein the mammalian fb41a receptor antagonist is not previously known.

110. A process for determining whether a chemical compound specifically binds to and activates a mammalian fb41a receptor, which comprises contacting cells producing a second messenger response and expressing on their cell surface the mammalian fb41a receptor, wherein such cells do not normally express the mammalian fb41a receptor, with the chemical compound under conditions suitable for activation of the mammalian fb41a receptor, and measuring the second messenger response in the presence and in the absence of the chemical compound, a change in the second messenger response in the presence of the chemical compound indicating that the compound activates the mammalian fb41a receptor.

111. The process of claim 110, wherein the second messenger response comprises chloride channel activation and the change in second messenger is an increase in the level of inward chloride current.

112. A process for determining whether a chemical compound specifically binds to and inhibits activation of a mammalian fb41a receptor, which comprises separately contacting cells producing a second messenger response and expressing on their cell surface the mammalian fb41a receptor, wherein such cells do not normally express the mammalian fb41a receptor, with both the chemical compound and a second chemical compound known to activate the mammalian fb41a receptor, and with only the second chemical compound, under conditions suitable for activation of the mammalian fb41a receptor, and measuring the second messenger response in the presence of only the second chemical compound and in the presence of both the second chemical compound and the chemical compound, a smaller change in the second messenger response in the presence of both the chemical compound and the second chemical compound than in the presence of only the second chemical compound indicating that the chemical compound inhibits activation of the mammalian fb41a receptor.

113. The process of claim 112, wherein the second messenger response comprises chloride channel activation and the change in second messenger response is a smaller increase in the level of inward chloride current in the presence of both the chemical compound and the second chemical compound than in the presence of only the second chemical compound.

114. A process of any one of claims 110, 111, 112 or 113, wherein the mammalian fb41a receptor is a human fb41a receptor.

115. The process of claim 114, wherein the human fb41a receptor has substantially the same amino acid sequence as encoded by the plasmid FB41a (ATCC Accession No. 209449).

116. The process of claim 114, wherein the human fb41a receptor has substantially the same amino acid sequence as that shown in FIGS. 2A-2C (Seq. I.D. No. 2).

117. The process of claim 114, wherein the human fb41a receptor has an amino acid sequence identical to the amino acid sequence shown in FIGS. 2A-2C (Seq. I.D. No. 2).

118. The process of any one of claims 110, 111, 112, 113, 114, 115, 116, or 117, wherein the cell is an insect cell.

119. The process of any one of claims 110, 111, 112, 113, 114, 115, 116, or 117, wherein the cell is a mammalian cell.

120. The process of claim 119, wherein the mammalian cell is nonneuronal in origin.

121. The process of claim 120, wherein the nonneuronal cell is a COS-7 cell, CHO cell, 293 human embryonic kidney cell, NIH-3T3 cell or LM(tk-) cell.

122. The process of claim 119, wherein the compound is not previously known to bind to a mammalian fb41a receptor.

123. A compound determined by the process of claim 122.

124. A pharmaceutical composition which comprises an amount of a mammalian fb41a receptor agonist determined by the process of claim 110 or 111 effective to increase activity of a mammalian fb41a receptor and a pharmaceutically acceptable carrier.

125. A pharmaceutical composition of claim 124, wherein the mammalian fb41a receptor agonist is not previously known.

126. A pharmaceutical composition which comprises an amount of a mammalian fb41a receptor antagonist determined by the process of claim 112 or 113 effective to reduce activity of a mammalian fb41a receptor and a pharmaceutically acceptable carrier.

127. A pharmaceutical composition of claim 126, wherein the mammalian fb41a receptor antagonist is not previously known.

128. A method of screening a plurality of chemical compounds not known to activate a mammalian fb41a receptor to identify a compound which activates the mammalian fb41a receptor which comprises:
(a) contacting cells transfected with and expressing the mammalian fb41a receptor with the plurality of compounds not known to activate the mammalian fb41a receptor, under conditions permitting activation of the mammalian fb41a receptor;
(b) determining whether the activity of the mammalian fb41a receptor is increased in the presence of the compounds; and if so
(c) separately determining whether the activation of the mammalian fb41a receptor is increased by each compound included in the plurality of compounds, so as to thereby identify the compound which activates the mammalian fb41a receptor.

129. A method of claim 128, wherein the mammalian fb41a receptor is a human fb41a receptor.

130. A method of screening a plurality of chemical compounds not known to inhibit the activation of a mammalian fb41a receptor to identify a compound which inhibits the activation of the mammalian fb41a receptor, which comprises:
(a) contacting cells transfected with and expressing the mammalian fb41a receptor with the plurality of compounds in the presence of a known mammalian fb41a receptor agonist, under conditions permitting activation of the mammalian fb41a receptor;
(b) determining whether the activation of the mammalian fb41a receptor is reduced in the presence of the plurality of compounds, relative to the activation of the mammalian fb41a receptor in the absence of the plurality of compounds; and if so
(c) separately determining the inhibition of activation of the mammalian fb41a receptor for each compound included in the plurality of compounds, so as to thereby identify the compound which inhibits the activation of the mammalian fb41a receptor.

131. A method of claim 130, wherein the mammalian fb41a receptor is a human fb41a receptor.

132. A method of any one of claims 128, 129, 130, or 131, wherein the cell is a mammalian cell.

133. A method of claim 132, wherein the mammalian cell is non-neuronal in origin.

134. The method of claim 133, wherein the non-neuronal cell is a COS-7 cell, a 293 human embryonic kidney cell, a LM(tk−) cell or an NIH-3T3 cell.

135. A pharmaceutical composition comprising a compound identified by the method of claim 128 or 129 effective to increase mammalian fb41a receptor activity and a pharmaceutically acceptable carrier.

136. A pharmaceutical composition comprising a compound identified by the method of claim 130 or 131 effective to decrease mammalian fb41a receptor activity and a pharmaceutically acceptable carrier.

137. A method of treating an abnormality in a subject wherein the abnormality is alleviated by increasing the activity of a mammalian fb41a receptor which comprises administering to the subject an amount of a compound which is a mammalian fb41a receptor agonist effective to treat the abnormality.

138. A method of claim 137, wherein the abnormality is a regulation of a steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a visceral innervation disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder, or migraine.

139. A method of treating an abnormality in a subject wherein the abnormality is alleviated by decreasing the activity of a mammalian fb41a receptor which comprises administering to the subject an amount of a compound which is a mammalian fb41a receptor antagonist effective to treat the abnormality.

140. A method of claim 139, wherein the abnormality is a regulation of steroid hormone disorder, an epinephrine release disorder, a gastrointestinal disorder, a cardiovascular disorder, an electrolyte balance disorder, hypertension, diabetes, a respiratory disorder, asthma, a reproductive function disorder, an immune disorder, an endocrine disorder, a musculoskeletal disorder, a visceral innervation disorder, a neuroendocrine disorder, a cognitive disorder, a memory disorder, a sensory modulation and transmission disorder, a motor coordination disorder, a sensory integration disorder, a motor integration disorder, a dopaminergic function disorder, an appetite disorder, obesity, a sensory transmission disorder, an olfaction disorder, a sympathetic innervation disorder, or migraine.
